(19) United States
(12) Patent Application Publication
(10) Pub. No.: US 2013/0177928 A1
(43) Pub. Date: Jul. 11, 2013

(54) NORMALIZATION OF PLATELET BIOMARKERS

(71) Applicants: Jon Peterson, Bellefonte, PA (US); Giannoula Klement, Boston, MA (US); Joseph Italiano, Brookline, MA (US); Sean Downing, Methuen, MA (US)

(72) Inventors: Jon Peterson, Bellefonte, PA (US); Giannoula Klement, Boston, MA (US); Joseph Italiano, Brookline, MA (US); Sean Downing, Methuen, MA (US)

(73) Assignees: THE NEWMAN-LAKKA CANCER FOUNDATION, Scottsdale, AZ (US); THE BRIGHAM AND WOMEN'S HOSPITAL, INC., Boston, MA (US); ORTHO-CLINICAL DIAGNOSTICS, INC., Rochester, NY (US)

(21) Appl. No.: 13/511,168

(22) PCT Filed: Nov. 23, 2010
(86) PCT No.: PCT/US10/57786

§ 371 Date: Feb. 19, 2013

Related U.S. Application Data
(60) Provisional application No. 61/263,686, filed on Nov. 23, 2009.

(51) Int. Cl. G01N 33/68; G01N 33/74

(52) U.S. Cl. CPC G01N 33/6887; G01N 33/74
USPC 435/7.92; 435/287.2

(57) ABSTRACT

Described herein are methods useful for normalizing any biomarker in platelets. This has application in any method in which one wishes to ascertain or compare the level of a biomarker, e.g., for diagnostic or prognostic methods relating to a biomarker of interest. Using such an approach can permit the assessment of disease status (e.g., angiogenic status) of an individual with less error than an expression value that is not normalized or that is normalized to total protein levels. Also provided are methods for selecting a normalizing protein for normalizing biomarkers in a sample, e.g., a platelet sample.

### CROSS-REFERENCE TO RELATED APPLICATIONS

This International application claims the benefit of priority under 35 U.S.C. §119(e) of U.S. Provisional Application No. 61/263,686, filed Nov. 23, 2009, the contents of which are incorporated herein by reference in their entirety.

### FIELD OF THE INVENTION

The field of the invention relates to the identification and use of surrogate markers of platelet number, platelet concentration or platelet volume.

### BACKGROUND

Platelets are small anucleate cellular fragments that play essential roles in hemostasis, repair of vascular damage and wound healing (Folkman, J. Ann. Rev. Med. 57 (2006) 1-18). Dr. Judah Folkman and colleagues described the phenomenon that endogenous angiogenesis-regulatory proteins were inside or associated with platelets (Folkman, J., et al. Thromb Haemost 86 (2001) 23-33). Several subsequent studies reported that circulating platelets in mice take up and sequester angiogenesis regulatory proteins when a microscopic tumor is present in a mouse (Klement, G. et al., Blood (ASH Annual Meeting Abstracts), 104 (2004) 839; Naumov, G. N. et al., J. Natl. Cancer Inst. 98 (2006) 316-325; Almog, N. et al., FASEB J. (2006)947-949).

Angiogenesis is the process of new blood vessel growth, which is essential in development, reproduction and repair. However, pathological angiogenesis occurs in tumor formation and many neoplastic diseases (Folkman, J. Ann. Rev. Med. 57 (2006) 1-18). Tumor cells release or induce the release of angiogenesis proteins, which stimulate the proliferation, migration, and tube formation of capillary endothelial cells (Hanahan, D., and Folkman, J. Cell 86 (1996) 353-354).

It has been proposed through a series of studies that angiogenesis regulatory proteins are exchanged locally at sites of platelet adhesion and aggregation (Klement, GL., et al., Blood, (2008) doi:10.1182/blood-2008-06-159541).

The levels of angiogenic proteins such as VEGF, bFGF, or PF-4 have been evaluated in serum and plasma as potential diagnostic markers (biomarkers) for early detection of disease. These approaches may be technically challenging due to the low concentration or short half-life of some of these biomarkers in circulation. Some angiogenic proteins in serum and plasma have been described to increase significantly in the presence of a large tumor mass.

In a study of paired serum and plasma samples, VEGF levels correlated with platelet count in 116 patients with colorectal cancer, but not in controls. These correlations were calculated indirectly by determining the differences between serum and plasma, which increased with disease progression. Additionally, the higher serum levels of VEGF in cancer patients were suggested to merely reflect platelet counts rather than tumor burden.

Thus, serum measurements cannot be assumed to include all of the analytes found in the platelets. Some platelet associated VEGF and bFGF, for example, may be released into the serum during agonist (thrombin) stimulation as encountered during serum clot formation, but significant levels remain associated with platelets and are presumably lost with the hematocrit (Åkerblom, B., et al. Upsala J Med Sci. 107(3) (2002) (165-171); Salgado, R., et al., Brit. J of Cancer, 80(5/6) (1999) 892-897).

### SUMMARY OF THE INVENTION

The methods described herein are based, in part, on the recognition that platelets actively sequester biomarkers such as angiogenic regulatory factors, and that changes in the level of biomarkers can provide early and sensitive indicators of diseases such as e.g., angiogenic diseases or disorders, including, among others, cancer. The methods described herein are also based, in part, on the observation that a normalized value for a biomarker (e.g., angiogenic regulator) measured in platelets is a better predictor of disease and/or angiogenic status than a value that is not normalized as the latter may only reflect platelet concentration. The closer the correlation between the platelet count and the normalization procedure, the better the ability to compare levels between samples for the diagnosis and monitoring of e.g., angiogenic disease.

The methods described herein relate to assessing the level or change in the level of a biomarker in a sample or samples from an individual, relative to the platelet count, platelet concentration or platelet volume, as determined from a normalization factor (e.g., actin).

In one aspect, for example, a method is described for assessing a biomarker level for a platelet preparation, the method comprising: (a) determining the level of a surrogate marker in a platelet preparation sample, wherein the surrogate marker corresponds to platelet number, platelet concentration or platelet volume; (b) determining the level of a biomarker in the sample, (c) normalizing the level of the biomarker in the sample to the level of the surrogate marker, whereby a normalized biomarker level for the sample is determined.

In one embodiment, the normalizing step comprises dividing the value obtained for the level of the biomarker in the sample by the value obtained for the level of the surrogate marker.

In another embodiment of this aspect, the surrogate marker is polymerized or monomeric actin. In another embodiment, the sample is placed under conditions that induce actin polymerization, such that actin in the sample is substantially polymerized.

In another embodiment of this aspect and all other aspects described herein, a high concentration of salt promotes actin polymerization.

While the methods can be employed to examine any biomarker, in one embodiment the biomarker is an angiogenic regulator.

In another embodiment, a change in the level of the biomarker is indicative of a disease state (e.g., angiogenic status or angiogenic disorder).

Also described herein is a method for identifying a surrogate marker for platelet number, platelet concentration, or platelet volume, the method comprising: (a) assaying the amount of a plurality of candidate markers in each sample of a series of samples prepared from a single platelet preparation according to a sampling factor; (b) comparing the amount of each candidate marker in each sample to the amount of candidate marker predicted according to the sampling factor, wherein the comparing step identifies the candidate marker of the plurality assayed in step (a) that has the closest correlation between the amount of candidate marker predicted and the amount of candidate marker measured, whereby the candidate marker is identified as a surrogate marker for platelet number, platelet concentration, or platelet volume.

In one embodiment of this aspect, the platelet preparation comprises lysed platelets.

In another embodiment of this aspect, the method further comprises testing the identified surrogate marker for variation under different physiological conditions.

In another aspect, methods are described herein for normalizing the amount of a biomarker in a sample, the method comprising normalizing the amount of a biomarker measured in a platelet preparation relative to a surrogate marker identified using the method comprising: (a) assaying the amount of a plurality of candidate markers in each sample of a series of samples prepared from a single platelet preparation according to a sampling factor; (b) comparing the amount of each candidate marker in each sample to the amount of candidate marker predicted according to the sampling factor, wherein the comparing step identifies the candidate marker of the plurality assayed in step (a) that has the closest correlation between the amount of candidate marker predicted and the amount of candidate marker measured, whereby the candidate marker is identified as a surrogate marker for platelet number, platelet concentration, or platelet volume.

In one embodiment of this aspect, the biomarker is an angiogenic protein.

In another embodiment of this aspect, the platelet preparation is obtained from a patient sample. In one embodiment, the method further comprises comparing a normalized level of the biomarker to a reference level to detect a change in the level of the biomarker in the patient sample.

Also described herein are methods for assessing a biomarker level for a platelet preparation, the method comprising: (a) placing a sample of isolated platelets obtained from the individual under conditions that induce actin polymerization, such that actin in the sample is substantially polymerized; (b) contacting the sample with an agent that selectively binds polymerized actin and detecting formation of a complex between the agent and polymerized actin, whereby the level of actin in the sample is measured; (c) measuring the level of a biomarker in the sample; (d) normalizing the level of the biomarker in the sample to the measured level of polymerized actin in the sample, whereby a normalized biomarker for the sample is determined.

Also described herein are methods for assessing a change in biomarker level of a sample, the method comprising: (a) placing a sample of isolated platelets obtained from the individual under conditions that induce actin polymerization, such that actin in the sample is substantially polymerized; (b) contacting the sample with an agent that selectively binds polymerized actin and detecting formation of a complex between the agent and polymerized actin, whereby the level of actin in the sample is measured; (c) measuring the level of a biomarker in the sample; (d) normalizing the level of the biomarker in the sample to the measured level of polymerized actin in the sample, (e) comparing a normalized level of the biomarker in the sample to a reference, wherein a difference in the normalized level of the biomarker compared to the reference indicates a change in the level of the biomarker of the individual.

In one embodiment, the agent that selectively binds polymerized actin comprises an antibody.

In one embodiment of this aspect, the conditions that induce actin polymerization comprise a high concentration of salt.

In another embodiment of this aspect, the biomarker is an angiogenic regulator.

In another embodiment of this aspect, a change in the level of the angiogenic regulator is indicative of a change in angiogenic state and/or the presence of an angiogenic disorder.

In another embodiment of this aspect, the angiogenic disorder is the presence of a tumor-associated disease.

In another embodiment of this aspect, the reference is obtained from biological samples obtained from a population of individuals. In one embodiment, each individual of the population is (substantially) free from an angiogenic disorder.

In another embodiment of this aspect, the reference is obtained from isolated platelets obtained from the individual at an earlier time point.

Also provided herein are kits for detecting a normalized level of at least one biomarker in platelets, the kit comprising: (a) at least one agent that selectively binds a platelet normalizing factor, (b) at least one agent that selectively binds a biomarker sequestered in platelets, and (c) packing materials and instructions for normalizing the level of at least one biomarker to the level of the normalizing factor.

Also provided herein are kits for detecting a normalized level of at least one biomarker in platelets, the kit comprising: (a) at least one reagent which, when contacted with an isolated platelet sample induces actin polymerization or depolymerization; and (b) an agent that selectively binds either (i) polymerized actin wherein the reagent of step (a) induces actin polymerization, or (ii) monomeric actin wherein the reagent of step (a) induces actin depolymerization; (c) an agent that binds a biomarker; and (d) packing materials and instructions for normalizing the level of the at least one biomarker to the level of polymerized or monomeric actin.

In one embodiment, the kit further comprises a solid support or a reagent that generates a detectable signal. In another embodiment, the kit further comprises a polymerized actin positive control. In another embodiment, the kit further comprises an agent that binds at least one other biomarker.

The kits described above can further comprise any one or more of the following: solid supports, reaction vessels, software for use with a detection system, sample holders etc.

Also provided herein are computer readable storage media having computer readable instructions recorded thereon to define software modules for implementing on a computer a method for assessing a biomarker level in a platelet sample, the computer readable storage medium comprising: (a) instructions for storing and accessing data representing a level of a biomarker and a level of a surrogate marker determined for a sample of isolated platelets obtained from at least one individual; (b) instructions for normalizing the level of the biomarker to the level of the surrogate marker via a normalization module, thereby producing a normalized level of the biomarker, (c) instructions for displaying retrieved content to a user, wherein the retrieved content comprises a normalized biomarker level.

In one embodiment, the computer readable storage medium further comprises instructions for comparing the normalized level of the biomarker to reference data stored on the storage device using a comparison module, whereby a change in the biomarker level is determined.

In another embodiment, the surrogate marker is polymerized or monomeric actin.

Also described herein are computer systems for obtaining data from a sample of isolated platelets obtained from at least one individual, the system comprising: (a) a specimen container to hold the sample; (b) a determination module configured to determine read-out information, wherein the read-out information comprises 1) information representing an amount of a surrogate marker of platelet number, platelet concentration or platelet volume, and 2) information representing an amount of a biomarker measured in the sample, (c) a storage device configured to store data output from the determination module, (d) a normalization module configured to normalize information representing a level of the biomarker to information representing an amount of the surrogate marker; (e) a display module for displaying retrieved content to the user, wherein the retrieved content comprises a normalized biomarker level.

In one embodiment, the computer system further comprises a comparison module adapted to compare the data obtained from the normalization module with reference data on the storage device, whereby a change in the level of the biomarker is determined.

In another embodiment, the surrogate marker is polymerized or monomeric actin.

In another aspect, the methods described herein relate to assessing a change in biomarker level in a platelet preparation, the method comprising: (a) contacting a platelet sample with an agent that selectively binds a normalizing protein and detecting the formation of a complex of the agent and the normalizing protein, whereby the level of the normalizing protein in the sample is measured; (b) measuring the level of a biomarker in the platelet sample; (c) normalizing the level of the biomarker in the sample to the level of the normalizing protein in the sample, and (d) comparing a normalized level of the biomarker in the sample to a reference, wherein a difference in the normalized level of the biomarker compared to the reference indicates a change in the level of the biomarker. In this and other aspects described herein, it is preferred, but not absolutely necessary that the platelet samples used to detect biomarker and normalization factor are obtained from dilutions of the same blood draw.

In one embodiment, the agent comprises an antibody.

Another aspect described herein relates to a method for assessing a change in angiogenic status of an individual, the method comprising: (a) placing a sample of isolated platelets obtained from the individual under conditions that induce actin polymerization, such that actin in the sample is substantially polymerized; (b) contacting the sample with an agent that selectively binds polymerized actin and detecting formation of a complex between the agent and polymerized actin, whereby the level of actin in the sample is measured; (c) measuring the level of an angiogenic regulator in the sample; (d) normalizing the level of the angiogenic regulator in the sample to the level of polymerized actin in the sample, (e) comparing a normalized level of the angiogenic regulator in the sample to a reference, wherein a difference in the normalized level of the angiogenic regulator compared to the reference indicates a change in the angiogenic status of the individual.

In one embodiment of this aspect and all other aspects described herein, a change in the angiogenic status is indicative of an angiogenic disorder.

In another embodiment of this aspect and all other aspects described herein, the angiogenic disorder is the presence of a tumor-associated disease.

In another embodiment of this aspect, the method further comprises administering an angiogenic modulator to the individual.

In another embodiment of this aspect and all other aspects described herein, the reference is obtained from biological samples obtained from a population of individuals. In one embodiment of this aspect, each individual of the population is free from an angiogenic disorder.

In another embodiment of this aspect and all other aspects described herein, the reference is obtained from isolated platelets obtained from the individual at an earlier time point.

In another embodiment of this aspect and all other aspects described herein, the conditions that induce actin polymerization comprise a high concentration of salt.

Also described herein is a method for treating an angiogenesis disorder in an individual, the method comprising: (a) placing a sample of isolated platelets obtained from the individual under conditions that induce actin polymerization, such that actin in the sample is substantially polymerized; (b) contacting the sample with an agent that selectively binds polymerized actin and detecting formation of a complex with polymerized actin, whereby the level of actin in the sample is measured; (c) measuring the level of an angiogenic regulator in the sample; (d) normalizing the level of the angiogenic regulator in the sample to the level of polymerized actin in the sample, (e) comparing a normalized level of the angiogenic regulator in the sample to a reference, wherein a difference in the normalized level of the angiogenic regulator compared to the reference is determined, indicating the presence of an angiogenic disorder; and (f) administering an angiogenic modulator to the individual, wherein the angiogenic disorder is treated.

In one embodiment of this aspect and all other aspects described herein, the angiogenic disorder is the presence of a tumor-associated disease. In another embodiment of this aspect and all other aspects described herein, the angiogenic disorder comprises a pre-angiogenic tumor.

In another embodiment of this aspect and all other aspects described herein, the reference is obtained from biological samples obtained from a population of individuals. In one embodiment of this aspect, each individual of the population is free from an angiogenic disorder.

In another embodiment of this aspect and all other aspects described herein, the reference is obtained from isolated platelets obtained from the individual at an earlier time point.

In another embodiment of this aspect and all other aspects described herein, the conditions that induce actin polymerization comprise a high concentration of salt.

Also described herein is a method for assessing a change in angiogenic status of an individual, the method comprising: (a) placing a sample of isolated platelets obtained from the individual under conditions that induce actin depolymerization, such that actin in the sample is substantially monomeric; (b) contacting the sample with an agent that selectively binds monomeric actin and detecting formation of a complex between the agent and the monomeric actin, whereby the level of actin in the sample is measured; (c) measuring the level of an angiogenic regulator in the sample; (d) normalizing the level of the angiogenic regulator in the sample to the level of monomeric actin in the sample, (e) comparing a normalized level of the angiogenic regulator in the sample to a reference, wherein a difference in the normalized level of the angiogenic regulator compared to the reference indicates a change in the angiogenic status of the individual.

In one embodiment of this aspect and all other aspects described herein, conditions that induce actin depolymerization comprise a low concentration of salt.

Also described herein is a method for treating an angiogenesis disorder in an individual, the method comprising: (a) placing a sample of isolated platelets obtained from the individual under conditions that induce actin depolymerization, such that actin in the sample is substantially monomeric; (b) contacting the sample with an agent that selectively binds monomeric actin and detecting formation of a complex between the agent and the monomeric actin, whereby the level of actin in the sample is measured; (c) measuring the level of an angiogenic regulator in the sample; (d) normalizing the level of the angiogenic regulator in the sample to the level of monomeric actin in the sample, (e) comparing a normalized level of the angiogenic regulator in the sample to a reference, wherein a difference in the normalized level of the angiogenic regulator compared to the reference is determined, indicating the presence of an angiogenic disorder; (f) administering an angiogenic modulator to the individual, wherein the angiogenic disorder is treated.

In one embodiment of this aspect and all other aspects described herein, the conditions that induce actin depolymerization comprise a low concentration of salt.

### DEFINITIONS

As used herein the phrase “conditions that induce actin polymerization” refers to a condition or set of conditions (e.g., temperature, pH, ionic strength, presence of buffers etc.) wherein actin is substantially polymerized in the platelet sample. Conditions that promote actin polymerization include e.g., heat, high ionic strength. Conversely, the phrase “conditions that induce depolymerization” refers to a condition or set of conditions wherein actin in the platelet sample is substantially in the monomeric form. Such conditions include e.g., low ionic strength, low temperature, or the presence of actin binding proteins or toxins.

As used herein the term “substantially polymerized” refers to conditions wherein at least 75% of the actin present in the sample exists in polymeric form; preferably at least 80%, at least 85%, at least 87%, at least 90%, at least 93%, at least 95%, at least 99%, or even 100% (i.e., all of the actin is polymerized) of the actin present in the sample is in the polymeric form.

As used herein, the term “substantially monomeric” refers to conditions wherein at least 75% of the actin present in the sample exists in monomeric form; preferably at least 80%, at least 85%, at least 87%, at least 90%, at least 93%, at least 95%, at least 99%, or even 100% (i.e., all of the actin is depolymerized) of the actin present in the sample is in the monomeric form.

As used herein the term “agent” refers to a protein-binding agent that permits detection and/or quantification of levels or expression levels for a normalizing protein (e.g., actin) in a sample. Such agents include, but are not limited to, antibodies, recombinant antibodies, chimeric antibodies, tribodies, midibodies, protein-binding agents, small molecules, recombinant protein, peptides, aptamers, avimers and protein-binding derivatives or fragments thereof.

As used herein, the term “selectively binds” means that an agent is selective for binding and/or complex formation with the polymerized form of actin, such that the amount of complexes of the agent with the monomeric form of actin are less than 30% of the total complexes formed, preferably less than 20%, less than 10%, less than 5%, less than 2%, less than 1% or even zero binding (i.e., no detectable binding to monomeric actin). Similarly, an agent that “selectively binds” the monomeric form of actin, forms complexes with polymeric actin at a rate less than 30% of the total complexes formed; preferably less than 20%, less than 10%, less than 5%, less than 2%, less than 1% or even zero binding (i.e., no detectable binding to polymeric actin). One skilled in the art can easily determine the selectivity of an agent using standard immunoassays including e.g., ELISA, and Western blotting, among others.

The term “sampling factor” as used herein refers to a known quantitative relationship of a sample to the preparation from which it is taken. A dilution factor used, for example, in a dilution series, is one non-limiting example of a sampling factor. Another non-limiting example of a sampling factor is repeated aliquots of a given volume to prepare a series of samples with a known number of aliquots, e.g., a first sample with 1 μl of preparation, a second sample with 2 μl of preparation, a third sample with 3 μl of preparation, etc. The series of samples set up in either of these ways will have predictable amounts of a given protein, where the prediction is based upon the measurement of the protein in the initial sample and the sampling factor.

As used herein, the phrase “a difference in the normalized level” refers to an increase or decrease in the level of a biomarker, e.g., angiogenic regulator, of at least 10% compared to a reference value. In one embodiment it is preferred that an increase in the level of an angiogenic regulator is at least 20%, at least 30%, at least 40%, at least 50%, at least 60%, at least 70%, at least 80%, at least 90%, at least 95%, at least 99%, at least 1-fold, at least 10-fold, at least 100-fold, at least 1000-fold or more higher than the reference level. In another embodiment, it is preferred that an decrease in the level of a biomarker, e.g., an angiogenic regulator, is at least 20%, at least 30%, at least 40%, at least 50%, at least 60%, at least 70%, at least 80%, at least 90%, at least 95%, at least 99%, or even 100% (i.e., absent) compared to a reference level. In an alternate embodiment, the “difference in the normalized level” refers to a statistically significant change (either an increase or decrease) in level of a biomarker, e.g., an angiogenic regulator, compared to a reference level.

As used herein, the term “biomarker” refers to a polypeptide expressed endogenously in an individual and found or sequestered in platelets. In one embodiment, the biomarker is an “angiogenic regulator.” The term “angiogenic regulator” is used throughout the specification as an example of a type of biomarker useful with the methods described herein. Similarly, an angiogenic disease or disorder is but one example of a condition associated with a biomarker as the term “biomarker” is used herein. The term “biomarker” does not encompass “surrogate markers” or “normalization factors” as those terms are used herein.

As used herein, the phrase “normalizing the level of the biomarker” or “normalizing the level of the angiogenic regulator” refers to the conversion of a data value representing the level of a biomarker (e.g., angiogenic regulator) in a sample by dividing it by the expression data value representing the level of a normalizing protein (e.g., actin) in the sample, thereby permitting comparison of normalized biomarker values among a plurality of samples or to a reference.

As used herein, the terms “normalizing protein”, “normalizing factor” and “surrogate marker” are used interchangeably herein and refer to a protein against which the amounts of a protein of interest are normalized to permit comparison of amounts of the protein of interest in different biological samples. Generally, a normalizing protein is constitutively expressed and is not differentially regulated between at least two physiological states or conditions from which samples will be analyzed, e.g., given disease and non-disease states. Thus, for example, a normalizing protein does not vary substantially outside of a range found in a normal healthy population (e.g., <30%, <25%, <20%, <15%, preferably <10%, <7%, <5%, <4%, <3%, <2%, <1% or less) or in the presence and absence of e.g., angiogenic disease. In one embodiment, a normalizing protein is selected based on the degree of correlation (e.g., lowest amount of scatter or lowest standard deviation among replicates) of the protein measured over a series of sample dilutions, compared to the predicted relationship of the dilution series (e.g., predicted by linear regression). In this embodiment, a normalizing protein is selected that has the closest degree of correlation (e.g., as compared to another protein in a protein sample subjected to the same measurement) between predicted protein levels and measured protein levels assessed over the dilution series. The term “closest degree of correlation” can refer to a standard deviation for protein measurements (e.g., replicate measurements) over a dilution series of less than 2 compared to the predicted relationship over the dilution series; preferably the standard deviation is less than 1.5, less than 1, less than 0.5, less than 0.1, less than 0.01, less than 0.001 or more, including a standard deviation of zero (e.g., measured and predicted values are the same). Alternatively, the “closest degree of correlation” can be assessed using confidence intervals (e.g., 90% CI, 95% CI, 99% CI etc.), which are known to those skilled in the art.

As used herein, the term “housekeeping gene” refers to a gene encoding a protein that is constitutively expressed, and is necessary for basic maintenance and essential cellular functions. A housekeeping gene generally is not expressed in a cell- or tissue-dependent manner, most often being expressed by all cells in a given organism. Some examples of housekeeping proteins include e.g., actin, tubulin, GAPDH, among others. In one embodiment, a housekeeping gene is used as a normalizing protein or surrogate marker of platelet count, platelet concentration or platelet volume.

As used herein, the phrase “high concentration of salt” refers to a solution comprising at least 50 mM salt (e.g., 50 mM NaCl, or 50 mM KCl).

As used herein, the phrase “low concentration of salt” refers to a solution comprising less than 40 mM salt; preferably less than 30 mM, less than 20 mM, less than 10 mM, less than 1 mM, less than 100 μM, less than 10 μM, less than 1 μM, or even 0 mM (i.e., no salt).

As used herein, the term “angiogenic modulator” refers to an agent that alters angiogenesis in an individual treated with the angiogenic modulator by at least 10% compared to the level of angiogenesis in an untreated individual. An angiogenic modulator can be an angiogenesis inhibitor or an angiogenesis activator. Within this context, at a minimum, an angiogenic regulator will have an effect on angiogenesis in the corneal micropocket assay as known in the art. In one embodiment angiogenesis is inhibited by at least 20%, at least 30%, at least 40%, at least 50%, at least 60%, at least 70%, at least 80%, at least 90%, at least 95%, at least 99%, or even 100% (i.e., absent) in an individual treated with an angiogenic modulator compared to an untreated individual. In an alternate embodiment, angiogenesis is increased by at least 10% in an individual treated with an angiogenic modulator compared to the level of angiogenesis in an untreated individual, preferably angiogenesis is increased by at least 20%, at least 30%, at least 40%, at least 50%, at least 60%, at least 70%, at least 80%, at least 90%, at least 95%, at least 99%, at least 1-fold, at least 2-fold, at least 5-fold, at least 10-fold, at least 50-fold, at least 100-fold, at least 500-fold, at least 1000-fold, at least 10000-fold or more in an individual treated with an angiogenesis activator compared to an untreated individual.

As used herein, the term “read-out information” refers to data derived from a signal indicating binding of an agent to or complex formation with a normalizing protein (e.g., polymerized actin); a signal can comprise e.g., light, fluorescence, colorimetric or other detectable signal that indicates agent binding to a normalizing protein.

As used herein, the term “agent that binds at least one angiogenic regulator” refers to a protein-binding agent that permits detection and/or quantification of levels or expression levels for an angiogenic regulator. Such agents include, but are not limited to, antibodies, recombinant antibodies, chimeric antibodies, tribodies, midibodies, protein-binding agents, small molecules, recombinant protein, peptides, aptamers, avimers and protein-binding derivatives or fragments thereof.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the'invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE FIGURES

FIGS. 1A and 1B are a series of graphs depicting correlation of actin to platelet count.

FIG. 2 is a graph showing PDGF levels in samples with varying levels of platelets, not normalized.

FIG. 3 is a graph showing normalized PDGF levels in samples with varying levels of platelets.

FIG. 4 is a graph showing the correlation of actin protein (μg) to platelet volume (μL).

FIG. 5 is a series of graphs showing the effect of correlating platelet count to actin (A), tubulin (B), and total protein (C).

FIG. 6 is a block diagram showing a system for assessing biomarker level in an individual using actin as an exemplary normalizing protein.

FIG. 7 is a block diagram showing exemplary instructions on a computer readable medium for assessing biomarker level in an individual using actin as a normalizing protein and can be applied, for example, to assessment of angiogenic status.

### DETAILED DESCRIPTION

Described herein are methods useful for normalizing any biomarker in platelets. This has application in any method in which one wishes to ascertain or compare the level of a biomarker, e.g., for diagnostic or prognostic methods relating to a biomarker of interest. Using such an approach can permit the assessment of disease status (e.g., angiogenic status) of an individual with less error than an expression value that is not normalized. Furthermore, normalizing expression or levels of a biomarker (e.g., angiogenic regulator) to a normalizing protein as described herein is more predictive of disease status (e.g., angiogenic status) than normalizing to total protein levels in a platelet sample. The methods described herein further relate to a method for selecting a normalizing protein for normalizing biomarkers, such as angiogenic regulators, in a sample, e.g., a platelet sample. Non-limiting examples of normalizing for measurements of angiogenic regulatory proteins are provided herein, as platelets scavenge and deposit these factors in disease states.

### Biomarkers

The methods described herein are useful for normalizing the amount of any biomarker present in a biological sample, e.g., in a preparation of platelets. In one embodiment, the biological sample comprises a cellular component containing actin. In one embodiment, the biomarker is an angiogenic regulator. The specification describes the methods in terms of an angiogenic regulator, however the methods are applicable with respect to any biomarker present in a biological sample, e.g., in a sample of platelets.

Non-limiting examples of angiogenic regulators are described in US Patent Application Nos. 20060134605 and 20060204951, which are exemplary of other published literature that detail such angiogenic regulators, and the contents of which are herein incorporated by reference in their entirety.

### Normalizing Proteins or Factors

Essentially any protein can be used as a normalizing protein, provided that the protein is constitutively expressed, and is not differentially regulated in disease states (e.g., angiogenic disease states) or in a disease state of interest. One of skill in the art can easily determine if a protein can be used as a normalizing protein by comparing the protein expression levels in samples taken at different time points from one individual, or among a plurality of samples taken from disease (e.g., cancer) and control populations. An appropriate normalization protein will not fluctuate widely (e.g., less than 30%) among time points or among disease populations.

In one embodiment, a normalizing protein is selected based on the degree of correlation determined for the normalizing protein measurements assessed over a series of diluted platelet samples. In this embodiment, a sample of platelets having a known platelet count is diluted into a series of samples (e.g., at least 3, 4, 5, 6, 7, 8, 9, 10, or more) using a dilution or sampling factor. The dilution series can represent e.g., a linear, exponential, or logarithmic relationship. A candidate normalizing protein, or a plurality of candidate proteins (e.g., at least two) are measured in each diluted platelet sample and the amount of each candidate protein in each sample is recorded. The data are e.g., plotted on a graph depicting the amount of protein measured at each platelet count and/or stored on a computer. In an embodiment where the dilution series represents a linear relationship, a linear regression analysis is performed. The measured protein amounts at each platelet dilution are compared to the predicted amounts based on the dilution factor and the degree of correlation of each protein is determined. For non-linear series, the appropriate predicted curve is determined based upon the sampling or dilution factor, and the data are compared to the predicted curve. In one embodiment, a normalizing protein having low scatter, preferably the lowest scatter relative to a plurality of other proteins similarly analyzed for scatter, is selected for use with the methods described herein. In another embodiment, a normalizing protein having close correlation between the predicted level and the measured level, preferably the closest correlation relative to a plurality of other proteins similarly analyzed, is selected for use with the methods described herein.

Some examples of potentially useful normalizing proteins include housekeeping genes (e.g., actin, tubulin, etc.). The platelet isoform of myosin, myosin HA, is another candidate for use in normalization. Spectrin, a cell surface actin associated protein, as well as other cytoskeleton associated proteins are also contemplated for use with the methods described herein. Proteins with a role in platelet budding are another category of normalization candidates for use with the methods described herein. A protein with a role in platelet budding refers to a protein involved in platelet formation from megakaryocyte cells. An exemplary protein involved in platelet budding includes, but is not limited to, platelet derived growth factor (PDGF). F-actin (also referred to herein as “polymerized actin”) is more amenable to measurement than some other proteins, since some other proteins have competing binding factors that interfere with the antibodies used and/or suffer from other sources of interference. Essentially any protein that reflects the number of platelets in normalization procedures described herein may be used. In one embodiment, the normalizing protein is actin.

P-Selectin cannot be used for normalization. Although it is a marker of vesicles in platelets, it is up-regulated during platelet activation and it is not present significantly on the cell surface. Tubulin and Total Protein measurements were considered as potential targets for normalization. However, surprisingly in direct measurements, actin was found to have a superior correlation.

In addition to proteins, other factors such as nucleic acid species can be used to normalize a biomarker. Platelets do not have a nucleus, but do carry various RNA species. The methods described herein also contemplate the detection of a level of such nucleic acids for use in normalization. Similar considerations also apply to the detection of a level of carbohydrate-based factors for use in normalization.

Methods and calculations for normalizing expression level data once a normalizing protein level is determined are known to those of skill in the art, and/or are described in the Examples herein.

### Inducing Actin Polymerization/Depolymerization

In one embodiment, the normalizing protein used with the methods described herein comprises actin. The actin or other normalizing protein will necessarily be present in platelets.

In one embodiment, protein or platelet samples are placed under conditions that induce actin polymerization. Such conditions include, but are not limited to, high ionic strength/high salt concentration, heat, etc. One of skill in the art can readily determine if a set of conditions (e.g., pH, temperature, salt concentration, etc.) induces actin polymerization by contacting a sample with an actin protein-binding agent that preferentially binds to polymerized actin and measuring binding using e.g., an Actin ELISA as described herein in the Examples section.

Actin is considered to be “substantially polymerized” if at least 75% of the actin present in the sample exists in polymeric form; preferably at least 80%, at least 85%, at least 87%, at least 90%, at least 93%, at least 95%, at least 99%, or even 100% (i.e., all of the actin is polymerized) of the actin present in the sample is in the polymeric form.

It is also contemplated herein that one can normalize to a non-polymerized form of actin (i.e., actin in a monomeric form) using an agent that selectively binds to monomeric actin for the measurement. Under this scenario, actin would be placed under conditions that favor depolymerization to the monomeric form prior to testing. In this embodiment, protein or platelet samples are placed under conditions that induce actin depolymerization (e.g., low salt solution). In order to detect the monomeric form of actin, it may be necessary to raise a monoclonal antibody to monomeric actin, as most actin antibodies are selective for the polymeric form. Without wishing to be bound by theory, this is likely due to formation of actin polymers upon injection and contact with physiological salinity (i.e., high salt), thus raising antibodies against polymerized actin. Raising an antibody against monomeric actin can be accomplished by e.g., immunizing mice with monomeric actin, which has been chemically blocked from polymerization. Monoclonal antibodies can be developed by conventional methods and screening for monomeric actin in e.g., a low salt solution.

Actin is considered to be “substantially monomeric” if at least 75% of the actin present in the sample exists in monomeric form; preferably at least 80%, at least 85%, at least 87%, at least 90%, at least 93%, at least 95%, at least 99%, or even 100% (i.e., all of the actin is depolymerized) of the actin present in the sample is in the monomeric form.

### Angiogenesis-Related Disorders

The methods described herein are useful in reducing the variance and improving the accuracy of early detection, diagnosis, and therapeutic treatment of, as one example, angiogenic diseases or disorders.

There are a variety of diseases or disorders in which angiogenesis is important. These diseases are referred to herein as angiogenic diseases or angiogenesis-related diseases. As used herein, the term “angiogenic disease or disorder” refers to a condition that is characterized by (or caused by) aberrant or unwanted, e.g. stimulated or suppressed, formation of blood vessels. Aberrant or unwanted angiogenesis may either cause a particular disease directly or exacerbate an existing pathological condition. Examples of angiogenic diseases include ocular disorders, e.g. diabetic retinopathy, macular degeneration, neovascular glaucoma, retinopathy of prematurity, corneal graft rejection, retrolental fibroplasias, rubeosis, retinal neovascularization due to intervention, ocular tumors and trachoma, and other abnormal neovascularization conditions of the eye, e.g., corneal neovascularization where neovascularization may lead to blindness.

Other angiogenic diseases or disorders that can be detected by measurement of differences in platelet factors include, but are not limited to, neoplastic diseases, e.g. tumors, including bladder, brain, breast, cervix, colon, rectum, kidney, lung, ovary, pancreas, prostate, stomach and uterus, tumor metastasis, benign tumors, e.g. hemangiomas, acoustic neuromas, neurofibromas, trachomas, and pyrogenic granulomas, hypertrophy, e.g. cardiac hypertrophy, inflammatory disorders such as immune and non-immune inflammation, chronic articular rheumatism and psoriasis, disorders associated with inappropriate or inopportune invasion of vessels, such as restenosis, capillary proliferation in atherosclerotic plaques and osteoporosis.

Angiogenesis has been associated with a number of different types of cancer, including solid tumors and blood-borne tumors. Solid tumors with which angiogenesis has been associated include, but are not limited to, cancer of the prostate, lung, breast, brain, ovarian, stomach, pancreas, larynx, esophagus, testes, liver, parotid, biliary tract, colon, rectum, cervix, uterus, endometrium, kidney, bladder and thyroid; as well as rhabdomyosarcomas, retinoblastoma, Ewing's sarcoma, neuroblastoma, and osteosarcoma, among others. Tumors in which angiogenesis is important include benign tumors such as acoustic neuroma, neurofibroma, trachoma, and pyogenic granulomas. Prevention of angiogenesis halts the growth of these tumors and the resultant damage to the animal due to the presence of the tumor. Angiogenesis is also associated with blood-borne tumors, such as leukemias, any of various acute or chronic neoplastic diseases of the bone marrow in which unrestrained proliferation of white blood cells occurs, usually accompanied by anemia, impaired blood clotting, and enlargement of the lymph nodes, liver and spleen. It is believed that angiogenesis plays a role in the abnormalities in the bone marrow and lymph nodes that give rise to lymphoma, myelodysplastic syndrome and multiple myeloma.

Stimulation of angiogenesis can benefit disorders involving collateral circulation where there has been vascular occlusion or stenosis (e.g. to develop a “biopass” around an obstruction of an artery, vein, or of a capillary system). Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangiitis obliterans, thrombotic disorders, vasculitis, and the like.

Other conditions or diseases that can be detected and/or treated or prevented with the methods described herein include, but are not necessarily limited to, heart attack (myocardial infarction) or other vascular death, stroke, death or loss of limbs associated with decreased blood flow, and the like. In addition, the methods described herein can be used to accelerate healing of wounds or ulcers; to improve the vascularization of skin grafts or reattached limbs so as to preserve their function and viability; to improve the healing of surgical anastomoses (e.g., as in re-connecting portions of the bowel after gastrointestinal surgery); and to improve the growth of skin or hair.

### Angiogenic Modulators

Within the methods of treatment of angiogenic disease described herein, as examples of therapeutic approaches for which the described normalizing methods can be applied, essentially any agent that modulates angiogenesis can be used. An agent can be a small molecule, an antibody, a receptor, a protein, a peptide, a nucleic acid, e.g., an aptamer or siRNA, or an endogenous molecule, among others. There is clearly overlap between the angiogenic regulators tested for and normalized using the methods described herein and angiogenic modulators that can be administered for the treatment of disease. However, for clarity, an “angiogenic regulator” is a polypeptide expressed endogenously in an individual and found or sequestered in platelets. An “angiogenic modulator” is an agent that, when administered exogenously, has an effect, positive or negative, on angiogenesis.

Some non-limiting examples of angiogenic modulators include, for example, VEGF inhibitors such as antibodies against VEGF (e.g., anti-VEGF) or antigenic epitopes thereof, and soluble VEGF receptors such as Flt-1, Flk-1/KDR, Flt-4, neuropilin-1 and -2; VEGF receptor inhibitors or antibodies against such receptors such as DC101 [ImClone Systems, Inc., NY]; tyrosine kinase inhibitors; prolactin; angiostatin; endostatin; somatostatin; protamine; interleukin-12; troponin-1; platelet factor 4; thrombospondin-1; interferon alpha; basic fibroblast derived growth factor (bFGF) inhibitors such as a soluble bFGF receptor; transforming growth factor beta; epidermal-derived growth factor inhibitors; platelet derived growth factor inhibitors; an integrin blocker; tissue inhibitors of metalloproteases such as TIMP1 and TIMP2; interferon-inducible protein 10 and fragments and analogs of interferon-inducible protein 10; peptide from retinal pigment epithelial cell; heparin octasaccharides; methionine aminopeptidase inhibitor; and tissue factor pathway inhibitor; vasostatin; calreticulin; IFN-α, -β and -γ; CXCL10; IL-4-12 and -18; osteopontin; restin; bevacizumab; carboxyamidotriazole;, TMP-470; suramin; SU5416; VEGF121; VEGF gs; VEGF 65; VEGF 89; bFGF; PDGF; angiopoietins; FGF-1; Ang-1; ephrin; plasminogen activators; matrix metalloproteinases; Dll-4; and thalidomide; among others.

### Angiogenesis Assays

Angiogenic modulators can be tested for efficacy by using an angiogenesis assay. For the avoidance of doubt, one can use any of a number of in vitro or in vivo angiogenesis assays to evaluate the influence of a given agent on angiogenesis. Whether or not a composition or formulation can treat or prevent diseases associated with an angiogenesis disorder can be determined by its effect in a mouse model. However, at a minimum, an angiogenic modulator as described herein will have anti-angiogenic activity in a HUVEC cell migration assay. Another useful assay for determining if the compositions and formulations as disclosed herein have anti-angiogenesis activity is the CAM assay, which is frequently used to evaluate the effects of angiogenesis regulating factors because it is relatively easy and provides relatively rapid results. An angiogenesis regulating factor useful in the methods and compositions described herein will modify the number of microvessels in the modified CAM assay described by Iruela-Arispe et al., 1999, Circulation 100: 1423-1431. The method is based on the vertical growth of new capillary vessels into a collagen gel pellet placed on the CAM. In the assay as described by Iruela-Arispe et al., the collagen gel is supplemented with an angiogenic factor such as FGF-2 (50 ng/gel) or VEGF (250 ng/gel) in the presence or absence of test agents. The extent of the angiogenic response is measured using FITC-dextran (50 μg/mL) (Sigma) injected into the circulation of the CAM. The degree of fluorescence intensity parallels variations in capillary density; the linearity of this correlation can be observed with a range of capillaries between 5 and 540. Morphometric analyses are performed, for example, by acquisition of images with a CCD camera. Images are then analyzed and imported into an analysis package, e.g., NHImage 1.59, and measurements of fluorescence intensity are obtained as positive pixels. Each data point is compared with its own positive and negative controls present in the same CAM and interpreted as a percentage of inhibition, considering the positive control to be 100% (VEGF or FGF-2 alone) and the negative control (vehicle alone) 0%. Statistical evaluation of the data is performed to check whether groups differ significantly from random, e.g., by analysis of contingency with Yates' correction.

Additional angiogenesis assays are known in the art and can be used to test angiogenic modulators for use with the methods described herein. These include, for example, the corneal micropocket assay, hamster cheek pouch assay, the Matrigel assay and modifications thereof, and co-culture assays.

Donovan et al. describe a comparison of three different in vitro assays developed to evaluate angiogenesis regulators in a human background (Donovan et al., 2001, Angiogenesis 4: 113-121, incorporated herein by reference). Briefly, the assays examined include: 1) a basic Matrigel assay in which low passage human endothelial cells (Human umbilical vein endothelial cells, HUVEC) are plated in wells coated with Matrigel (Becton Dickinson, Cedex, France) with or without angiogenesis regulator(s); 2) a similar Matrigel assay using “growth factor reduced” or GFR Matrigel; and 3) a co-culture assay in which primary human fibroblasts and HUVEC are co-cultured with or without additional angiogenesis regulator(s), the fibroblasts produce extracellular matrix and other factors that support HUVEC differentiation and tubule formation. In the Donovan et al. paper the co-culture assay provided microvessel networks that most closely resembled microvessel networks in vivo. However, the basic Matrigel assay and the GFR Matrigel assay can also be used by one of skill in the art to evaluate whether a given angiogenic modulator is an angiogenesis-inhibiting agent as necessary for the methods described herein.

Finally, an in vitro angiogenesis assay kit is marketed by Chemicon (Millipore). The Fibrin Gel In Vitro Angiogenesis Assay Kit is Chemicon Catalog No. ECM630. Other angiogenesis assays are disclosed in International Application No: WO2003/086178 and U.S. Patent Applications US2005/0203013 and US2005/0112063, and involve assaying endothelial cells on a permeable substrate (e.g., a collagen coated inserts of “Transwells”), contacting the assay with a test compound (e.g., a fumagillol derivative block copolymer conjugate), treating the assay with a marker (e.g., FITC label) and a permeability-inducing agent (e.g., vascular endothelial growth factor (VEGF) and platelet-activating factor (PAP) among others), and measuring the rate of diffusion of the marker compare to control.

### Dosage and Administration

In one aspect, the methods described herein provide a method for treating an angiogenesis-associated disease in a subject. In one embodiment, the subject can be a mammal. In another embodiment, the mammal can be a human, although the approach is effective with respect to all mammals. The method comprises administering to the subject an effective amount of a pharmaceutical composition comprising an angiogenic modulator, in a pharmaceutically acceptable carrier.

The dosage range for the agent depends upon the potency, and includes amounts large enough to produce the desired effect, e.g., a reduction in neovascularization in a tumor site or elsewhere. The dosage should not be so large as to cause unacceptable adverse side effects. Generally, the dosage will vary with the type of angiogenic modulator used (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in neovascular formation, number of blood vessels etc. (see “Efficacy Measurement” below). Such effective amounts can be gauged in clinical trials as well as animal studies for a given angiogenic modulator.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. For the treatment of tumors, the agent can be administered systemically, or alternatively, can be administered directly to the tumor e.g., by intratumor injection or by injection into the tumor's primary blood supply.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an angiogenic modulator can be targeted to tissue- or tumor-specific targets by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an angiogenic modulator permits the agent attached to accumulate additively at the desired target site. Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

In some embodiments, an angiogenic modulator may be combined with one or more agents such as chemotherapeutic or anti-angiogenic agents, for the treatment of an angiogenesis associated disease.

### Pharmaceutical Compositions

Methods described herein involve therapeutic compositions useful for treating an individual having an angiogenesis-related disease. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Also contemplated are pharmaceutical compositions with active RNAi ingredients in a preparation for delivery, or in references cited and incorporated herein in that section. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. Therapeutic compositions useful with the methods described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Examples of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

### Efficacy Measurement

The efficacy of a given treatment for an angiogenesis-associated disease can be determined by the skilled clinician. However, a treatment is considered “effective treatment,” as the term is used herein, if any one or all of the signs or symptoms of, as but one example, ocular neovascular disease or tumor vascularization are altered in a beneficial manner, other clinically accepted symptoms or markers of disease are improved, or even ameliorated, e.g., by at least 10% following treatment with an angiogenic modulator. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of new blood vessels; or (2) relieving the disease, e.g., causing regression of symptoms, reducing the number of new blood vessels in a tissue exhibiting pathology involving angiogenesis (e.g., the eye or a tumor site); and (3) preventing or reducing the likelihood of the development of a neovascular disease, e.g., tumor).

An effective amount for the treatment of a disease means that amount which, when administered to a mammal in need thereof, is sufficient to result in effective treatment as that term is defined herein, for that disease. Efficacy of an agent can be determined by assessing physical indicators of, for example cancer or ocular neovascular disease, such as e.g., visual problems, new blood vessel invasion, rate of vessel growth, angiogenesis, tumor growth rate etc, or tumor vascularization.

### Systems

Embodiments of the invention also provide for systems (and computer readable media for causing computer systems) to perform a method for normalizing the expression value of a biomarker (e.g., angiogenic regulator).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs, that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The normalization systems described herein include, in one aspect, a normalization module (10), the normalization module comprising: a determination system module (20) with computer-executable instructions for reading or receiving input detectable (platelet) reference signal from a sample, e.g., fluorescence signal from an agent that binds polymerized actin, computer-executable instructions for reading or receiving input detectable signal for a (platelet) biomarker and computer-executable instructions for output of read or received signal values (40) to a comparison module; a comparison module (80) with computer-executable instructions for receiving data from the determination system (or from an intermediate storage device (30), computer-executable instructions for comparing biomarker signal to reference marker signal (e.g., for polymerized actin) to generate a normalized biomarker value and computer-executable instructions for output of a normalized biomarker value to a display module; and a display module (110) with computer-executable instructions for receiving a normalized biomarker value from comparison module (80) and for display of the normalized value (100) on a display device (120) or other output (130; e.g., printer, output to a network interface, etc.). Comparison module (80) can also include computer-executable instructions for output of comparison results to a storage device or to a network interface.

Determination system (20) can include hardware (50) for detecting signal, e.g., a fluorescence signal detector, absorbance or transmission signal detector (e.g., a UV, IR or other light signal detector), radioisotope signal detector, flow cytometry signal, FACS signal, fluorescence microscopy signal, ELISA signal, Western blot signal, etc.). In one embodiment, the hardware (50) comprises a microtiter plate reader, also referred to as a microplate reader. The determination system has computer-executable instructions to provide, e.g., fluorescence information from a microplate reader (50) in computer-readable form.

Comparison module (80) can also comprise a further comparison sub-module with instructions for receiving and comparison of one normalized biomarker value to another (normalized to the same reference marker) to produce a result indicating the difference in normalized platelet biomarker values between two different measured samples. The comparison sub-module can further include instructions for output of a difference in normalized platelet biomarker values between two measured samples to a display module (110) or storage device (30).

The functional modules can be executed on one or multiple computers (90) or by using one or multiple computer networks.

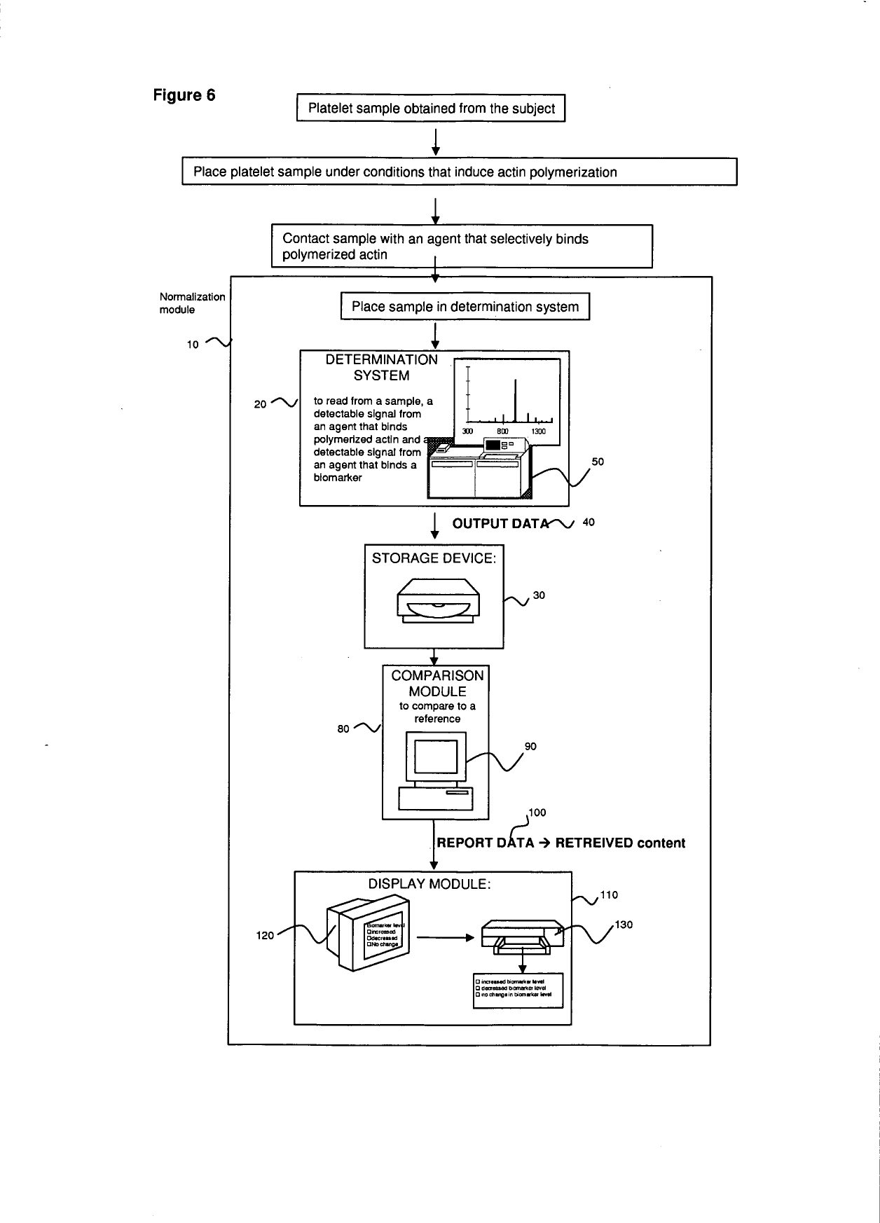

In one embodiment (see FIG. 6 for a schematic), a platelet sample obtained from a subject is placed under sample conditions that induce actin polymerization, e.g., in vitro in a sample vessel such as a test tube or well of a microtiter plate. In this embodiment, sample having substantially polymerized actin is contacted with an agent, such as an antibody, that selectively binds polymerized actin. The sample, with the agent, is then placed into or subjected to a determination system under control of a normalization module as described herein above to provide a normalizing reference value. The sample, a parallel sample from the same subject, or an aliquot of the same sample is also subjected to determination of a signal for a different biomarker of interest, e.g., an angiogenic biomarker. Normalization and comparison modules generate a value for that biomarker in the sample, normalized to the reference biomarker for that sample, e.g., polymerized actin. A comparison sub-module can compare values for a biomarker of interest from two different samples, normalized to reference biomarker, to provide an output of the difference in normalized biomarker levels between two or more samples.

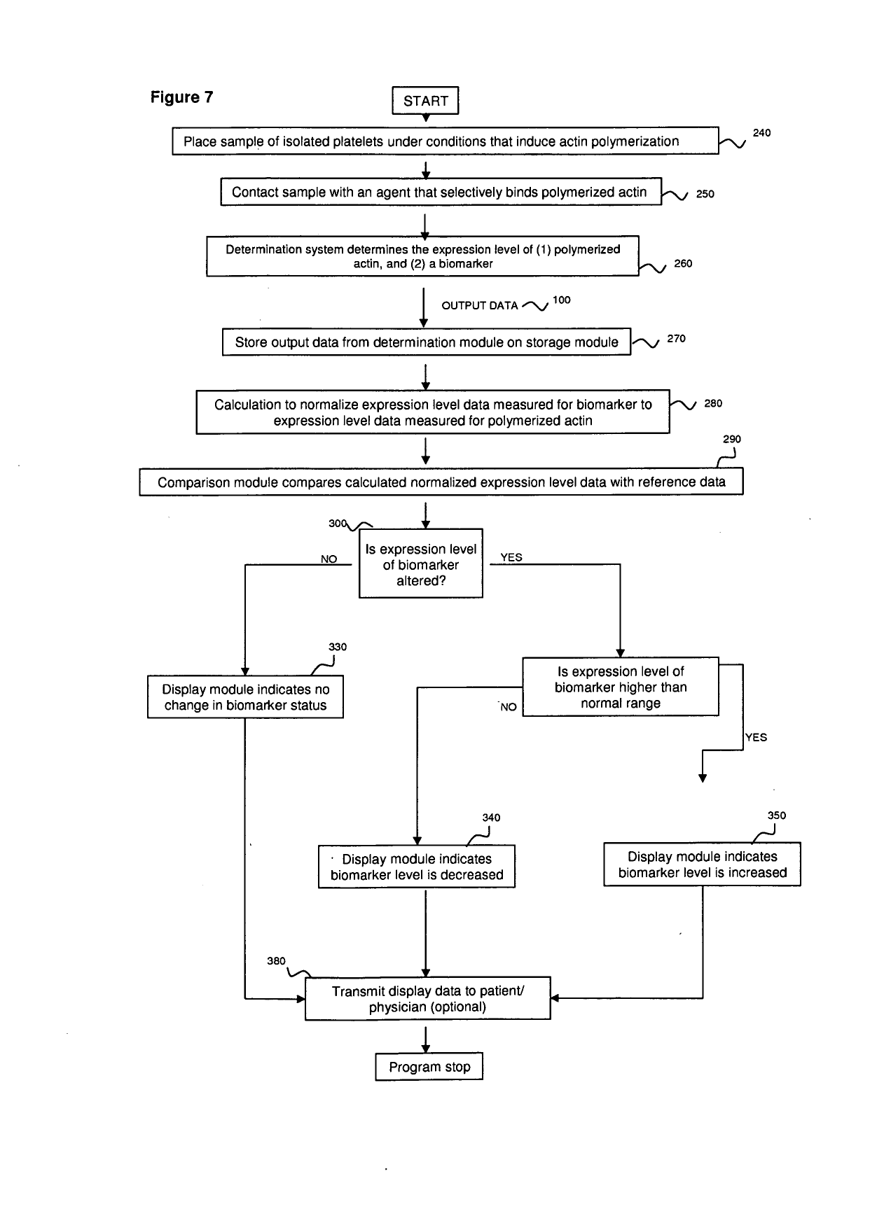

FIG. 7 shows a schematic flow chart of one embodiment of a system or routine as described herein in which one or more platelet biomarkers of interest are measured, normalized to a reference, and compared with normalized biomarker levels in another sample to generate an indication of the relative biomarker levels (or change in the biomarker level of interest) between samples. The routine includes: step (240), of placing isolated platelets under conditions that induce actin polymerization; step (250), of contacting the sample with an agent that selectively binds polymerized actin; step (260) of determining the expression level of polymerized actin and another biomarker of interest; at step (100), data from the determination system are output and can be (270) stored via a storage module; step (280), of calculation to normalize expression level data for biomarker of interest to expression level data measured for polymerized actin; step (290) of comparing normalized expression level data with reference data (e.g., from another sample or from a standard). An output or display routine from a comparison module determines (300) whether expression level of a biomarker of interest is altered. If yes, the routine determines whether the expression level of the biomarker of interest is higher than normal range. If yes, the display module (routine element (350)) indicates that biomarker of interest is increased, optionally further transmitting this information (via routine element 380) to a user, e.g., a physician or patient. If expression level of the biomarker of interest is altered, and the level is lower than normal range, the display module (routine element (340)) indicates that biomarker of interest is decreased, optionally further transmitting this information (via routine element (380)) to a user. If biomarker of interest is not altered, the display module indicates (via routine element (330) no change in biomarker status, optionally further transmitting this information (via routine element (380)) to a user.

The determination system (20), can comprise any system for detecting a signal from a protein binding agent. Such systems can include flow cytometry systems, fluorescence assisted cell sorting systems, fluorescence microscopy systems (e.g., fluorescence microscopy, confocal microscopy), any ELISA detection system and/or any Western blotting detection system.

The information determined in the determination system can be read by the storage device (30). As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon expression level or protein level information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression level information.

In one embodiment the reference data stored in the storage device to be read by the comparison module is chromogenic data or fluorescence emission data obtained from an ELISA determination system.

The “comparison module”, and computer readable instructions thereof can use a variety of available software programs and formats for the comparison operative to compare fluorescence data determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, and may be optimized for particular data comparisons that are conducted. The comparison module provides computer readable information related to normalized expression level of an angiogenic regulator, angiogenic status of an individual, efficacy of treatment in an individual, and/or method for treating an individual.

The comparison module, or any other module of the invention, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment of the present invention, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user to provide a content based in part on the comparison result that may be stored and output as requested by a user using a display module.

The content based on the comparison result, may be a normalized expression value compared to a reference showing the angiogenic status an individual.

In one embodiment of the invention, the content based on the comparison result is displayed on a computer monitor. In one embodiment of the invention, the content based on the comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The present invention therefore provides for systems (and computer readable media for causing computer systems) to perform methods for assessing the angiogenic status of an individual.

Systems and computer readable media described herein are merely illustrative embodiments of the invention for performing methods of assessing angiogenic status in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

### Materials and Methods Useful for Measuring and Normalizing Platelet Protein Levels

### Actin ELISA Materials and Solid Phase Coating:

Detection antibody: (Millipore/Chemicon) murine monoclonal MAB1501R), Biotin conjugated at Ortho Clinical Diagnostics diluted to a working strength of 800 ng/mL in RD. The antibody was biotinylated utilizing standard methods and described below.

Capture antibody (Millipore/Chemicon) MAB1501R, murine monoclonal, 100 μg/vial High binding microwell plates (CoStar cat#2592) were coated with 100 μl coating antibody solution containing 2 μg/mL antibody in BuPH buffer pH 7.2 (Pierce 28372), incubated overnight in high humidity, washed with wash buffer (as described below) three times with 400 μL per well. The plates were then post-coated with 1504/well of Starting Block (Pierce 37542) to each well, incubated at room temperature in a humid box for a minimum of 2 hours, aspirated (not washed) 1×2 seconds, allowed to dry in a humidity controlled incubator and pouched in a sealed bad with a dessicant and stored at 2-8° C. until use.

### Biotinylations

Briefly, the antibody was mixed with Biotin-LC-LC-NHS (Pierce) dissolved in Dimethylformamide (Sigma) at a ratio of 1:10 (Antibody:biotin) for two hours at 20° C. Glycine was added to the antibody/biotin mixture at a ratio of 200:1 (glycine:biotin) and mixed for 15 minutes at 20° C. The antibody-biotin conjugate was exchanged into 0.1 M Phosphate, 0.3 M NaCl pH 6.0 buffer with a Nap-5 column (GE Healthcare). The antibody-biotin conjugate was diluted in Reagent Diluent to working strength and stored at 4° C.

### Actin ELISA Calibrators

Non-muscle Actin purified from human platelet, (Cytoskeleton, INC part APHL95) 1 mg vial reconstituted in 1 mL water, sub-aliquoted into single use vials and stored frozen at −70 until use. Calibrator levels were prepared by diluting the actin in Polymerization Buffer: (10 mM Tris, pH 7.5, 2 mM MgCl2 and 50 mM KCl); then diluted to 1000 ng/mL and then serial dilutions in Polymerization Buffer to 31 ng/mL for 6 calibrator levels plus a level 0 (Polymerization Buffer, diluent)

Other Reagents for ELISA Testing and/or Development.
Streptavidin-HRP (R&D Systems DuoSet generic reagent Part 890803), diluted 1:200 in Reagent Diluent.
20× Wash Buffer Concentrate (Ortho Clinical Diagnostics part 933730) diluted in deionized water
TMB Peroxide Substrate for ELISA (Moss, Inc part # TMBE-1000) used undiluted
4N Sulfuric Acid (Ortho Clinical Diagnostics part 933040)
Actin Polymerization Biochem Kit (Cytoskeleton, INC, Cat #BK003)

### Equipment

Autowash 95 Microplate washer (Ortho Clinical Diagnostics) Calibrated and volume verified prior and during use.
Shaker Incubator (Ortho Clinical Diagnostics, Chelsea type), heated orbital shaker with a 1 mm rotation at 600 rpm.
Rainin manual pipettors, all calibrated and volume verified prior to and during use: single channel: L20, L200, L300, L1000, L5000; multichannel L300
Sunrise Microplate reader (Tecan) with filters at 450 and 620 nm
Magellan microplate reader software (Tecan, ver 5)
Sorvall Legend RT centrifuge.
Beckman Coulter LH755 Analyzer for CBC platelet counting.
Beckman Airfuge (Ultracentrifuge)
BioTek Synergy 2 Fluorescence spectrophotometer: Ex(360/40)/Em (420/50)

### Human Platelet and Plasma (Platelet Poor) Preparation

Healthy Controls.

Venous blood samples were, also collected from 64 presumably healthy volunteers, comprised of samples obtained prospectively from colonoscopy screening patients at Mayo Clinic, Rochester, Minn. as well as employees of Children's Hospital Boston. All collections were performed after obtaining informed consent in accordance to institutional practice and guidelines.

Patients.

Peripheral venous blood samples were collected from patients with histologically diagnosed cancer admitted to the Massachusetts General Hospital of the Dana Farber Cancer Institute. All collections were performed after obtaining informed consent in accordance to institutional practice and guidelines.

The samples were processed according to standard methods for platelet collection. Briefly, whole blood was drawn by venipuncture into a vacuum tube containing 105 mM citrate (pH 5) anticoagulant at a ratio of 1:9 (vol/vol) buffer to blood. The tubes were inverted to mix the blood and anticoagulant and kept at ambient temperature throughout the processing. This was to avoid activation of the platelets with loss of the contents if stored with refrigeration. The blood samples were centrifuged for 20 minutes at 150×g using a Sorval swinging-bucket rotor. Following the first centrifugation, 1 mL of the top phase (platelet rich plasma, or PRP) was transferred into each of as many Eppendorf tubes as were required (typically 2 tubes) and centrifuged for 10 minutes at 900×g. The supernatant comprised of platelet poor plasma (or PPP) was transferred to another tube and the residual plasma blotted away from the inside walls of the tube containing the platelet pellet. The platelet and plasma samples were then stored at −80° C. until analysis.

### Platelet Lysis

Platelet samples were thawed and 100 μl of lysis buffer, containing 0.5% Triton X-100 (Fluka) and Protease Inhibitor cocktail (Sigma P8340) in PBS buffer pH 7.2 (Pierce), was added to each platelet pellet. The platelet pellet membranes were solubilized with the lysis buffer, pipetted up and down and vortexed until mostly translucent; 1.5 ml of PBS buffer was then added to each lysed platelet sample, yielding a 16× platelet lysate solution, which was diluted for analysis as described below.

The protease inhibitor cocktail (Sigma item # P8340) was provided as a concentrate which required a 1:100 dilution and contained the following inhibitors: AEBSF [4-(2-Aminoethyl)benzenesulfonyl fluoride hydrochloride] at 104 mM for serine proteases; Aprotinin at 0.085 mM for serine proteases; Bestatin hydrochloride at 4 mM for aminopeptidases; E-64, [N-(trans-Epoxysuccinyl)-L-leucine 4-guanidinobutylamide] at 1.4 mM for cysteine proteases; Leupeptin hemisulfate salt at 2 mM for serine and cysteine proteases; Pepstain A at 1.5 mM for acid proteases.

### Platelet Lysate Dilution Factors

The dilution factor of the 16× platelet lysate was 32 fold in polymerization buffer.

### Example 1

Serum measurements cannot be assumed to include all of the analytes found in the platelets. Some platelet associated VEGF and bFGF, for example, may be released into the serum during agonist (thrombin) stimulation as encountered during serum clot formation, but significant levels remain associated with platelets and are presumably lost with the hematocrit (Åkerblom, B., et al. Upsala J Med Sci. 107(3) (2002) (165-171); Salgado, R., et al., Brit. J of Cancer; 80(5/6) (1999) 892-897).

With this as a perspective and in order to determine if platelets selectively scavenge angiogenesis regulatory proteins e.g., from a tumor, it is important to first be able to isolate the platelet from whole blood without activation and spilling of the contents of the platelet. Secondly, it is important to be able to enumerate the number of platelets in a given sample under analysis in order to normalize the measured level of protein to the number of platelets, rather than a result that simply reflects the number of platelets. The normalization methods described herein provide normalization without requiring platelet count to be determined.

Direct CBC (Complete Blood Count) methodologies are typically performed to enumerate platelets and determine their volume. However, CBC measurements cannot be performed on platelet pellet samples, which is the most preferred platelet isolation method. Poor correlations exist between the whole blood CBC platelet counts and the levels found in stored platelet samples, as shown herein. It should be noted that CBC methods are prone to error and in fact may be largely discrepant, based on the instrument (Pieter, F. et al., Transfusion, 49 (2009) 81-90). In order to enumerate platelets in pellet samples, a method was developed to measure and identify a surrogate marker to enumerate platelets. In practice, it is demonstrated that a factor such as actin can be used to normalize platelet samples without the need to count the platelets.

### Results/Discussion: Normalization

As described herein, angiogenesis regulatory protein results obtained from platelets could reflect either the levels found due to “scavenging” from diseased tissues or merely the number of platelets in a given sample. Since the aim of this study was to determine the levels of specific proteins in platelets which could be used as a diagnostic approach to detect angiogenesis diseases, including cancer, it was important to develop a means to enumerate the number of platelets in a given sample.

### Estimates of Platelet Counts, Based on Index CBC Data

One method was tested to determine if the number of platelets in a platelet pellet sample could be estimated based on platelet count obtained by CBC (Complete Blood Count, flow cytometry) performed in the clinical lab with a different plasma sample from the same individual.

In order to test this, whole blood samples were obtained from twenty six (26) non-diseased individuals in both EDTA and citrate vacuum phlebotomy tubes. Because the typical CBC test is performed with EDTA plasma and the platelets are prepared in citrate, this was determined to be the closest approximation to the suggested approach. It was recognized that the gating of the CBC analyzer may have under estimated the number of platelets in the citrated plasma PRP.

The group of 26 individuals consisted of 19 females and 7 males with an average age of 48 years+/−7 years (1SD) and an age range of 33 to 61. CBC platelet counts were obtained from the EDTA whole blood, the citrated platelet rich plasma (PRP) and the resultant platelet poor plasma (PPP) obtained after centrifugation of 1 mL PRP and isolation of the platelet pellet (Table 1). The difference between the PRP and PPP was calculated to be number of platelets in the pellet. The platelet counts obtained from whole blood were typically lower than those obtained from PRP. Without wishing to be bound by theory, this result may reflect that platelets tend to have greater buoyancy compared to the rest of the hematocrit. It was discovered that the degree of error which would be introduced by this method would be with a positive bias of ˜57,000 with a range of 221,000 to 542,000 based on the 95% confidence intervals.

TABLE 1
CBC platelet counts obtained from whole blood, and
sub-fractions from 26 individuals

| ID | WB CBC | PRP CBC |
|---|---|---|
| 1 | 386 | 482 |
| 2 | 334 | 406 |
| 3 | 270 | 365 |
| 4 | 245 | 289 |
| 5 | 296 | 335 |
| 6 | 303 | 239 |
| 7 | 274 | 300 |
| 8 | 317 | 432 |
| 9 | 210 | 230 |
| 10 | 225 | 254 |
| 11 | 244 | 337 |
| 12 | 326 | 417 |
| 13 | 258 | 276 |
| 14 | 278 | 304 |
| 15 | 280 | 265 |
| 16 | 307 | 293 |
| 17 | 500 | 421 |
| 18 | 249 | 350 |
| 19 | 232 | 309 |
| 20 | 352 | 509 |
| 21 | 366 | 495 |
| 22 | 233 | 274 |
| 23 | 382 | 401 |
| 24 | 181 | 221 |
| 25 | 379 | 499 |
| 26 | 355 | 542 |
| Avg | 299 | 356 |
| SD | 70 | 96 |
| 95% CI LB | 181 | 221 |
| 95% CI UB | 500 | 542 |

### Example 2

### Normalization with Actin Measurements

A structural platelet protein that is constitutively expressed, and not differentially regulated in most disease states, was measured and determined whether it would be a desirable ELISA target for normalization. Several candidate targets were evaluated and tested (data not shown), and a desirable candidate was determined to be actin. Direct measurements of CBC enumerated platelet preparations with Actin, Tubulin and Total Protein were performed and the correlations to platelet counts were found to be superior with actin as shown here in Table 2.

Actin in platelets exists in a dynamic monomer-polymer equilibrium, which relates to its function (Italiano J. E. et al. Platelets in Hematologic and Cardiovascular Disorders, Cambridge University Press, New York, 2008, pp. 1-20). In an ELISA it is useful to understand this polymer/monomer equilibrium in order to control it (in vitro) for accurate measurements. It is also useful to be able to convert actin and/or maintain the actin form as either monomer or polymer.

TABLE 2
Comparison of enumerated platelet preparations

| Platelet Count | Actin | Tub | Avg Tot |
|---|---|---|---|
| (by CBC) | μg/mL | ng/mL | Prot. |
| 33 | 0.7 | 27 | 1003 |
| 58 | 0.5 | 23 | 667 |
| 66 | 2.4 | 34 | 1347 |
| 89 | 3.3 | 29 | 830 |
| 115 | 1.0 | 52 | 1166 |
| 129 | 7.4 | 36 | 1162 |
| 141 | 0.9 | 19 | 624 |
| 173 | 2.2 | 57 | 1470 |
| 178 | 10.1 | 31 | 1170 |
| 199 | 10.6 | 39 | 755 |
| 258 | 14.7 | 45 | 1396 |
| 282 | 6.4 | 58 | 1301 |
| 397 | 19.1 | 35 | 1431 |
| 596 | 28.8 | 66 | 1879 |
| Correlation (R\hat{ }2) to Platelet Count | 0.865 | 0.404 | 0.499 |

### Actin Polymerization

Actin physically exists in equilibrium between monomeric and polymeric forms, which relates to its biological function. Polymeric actin is also referred to herein as F-Actin. Without wishing to be bound by theory, F-actin can be used with the methods described herein because it reflects platelets with effective sequestration methods. Methods were tested to effectively and reproducibly measure the levels of actin by controlling the equilibrium towards one form or another.

The level of polymerization driven by buffer conditions was made possible by the use of an actin polymerization assay (Cytoskeleton, Inc. BK003) according to the manufacturer's instructions. In general, the stacking and interaction of the pyrene actin, which occurs with polymerization, allows measurement of fluorescence, which increases with polymer length. The fluorescence data were collected on a BioTek Synergy 2 Fluorescence spectrophotometer with the following filters: Excitation (360/40) and Emission (420/50). The top probe vertical offset was set to 7 mm and the optics position was set to Top 50%; the BioTek contained a tungsten light source and the samples were prepared and tested in a standard black 96 well plate. As will be apparent to one skilled in the art, alternative assays and measurement techniques to assay for alternative candidates may be employed. The particular techniques employed in these demonstrative examples are illustrative and are not intended to be limiting.

The pyrene labeled muscle actin and buffers provided in the kit were prepared according to the manufacturer's instructions. Briefly, pyrene actin was thawed, placed on ice in G-buffer (low ionic strength buffer that drives actin towards a monomer (globular) form) was added to each tube (final concentration 0.4 mg/mL). The pyrene actin was incubated on ice in the dark for one hour to depolymerize any actin oligomers. Buffers and additives were tested for their effect on actin (de)polymerization. Two wells of each of the following three controls were run along with the two wells containing the test chemical/protein: G-buffer alone, G-buffer with pyrene actin and G-buffer with pyrene actin and 20 μL of test buffer (i.e., the buffer with additives). After reading for baseline, either 20 uL of control (G) buffer or 20 uL of test buffer were added to the pyrene actin/G-buffer wells; the fluorescence data (Ex 360/Em 420) were collected every minute for 20 minutes. After this initial reading, 20 uL of the 10× actin polymerization buffer (provided by Cytoskeleton) was added to all eight wells and data was collected every minute for an additional 40 minutes or until the fluorescence signal reached a plateau.

Conditions that promoted monomer and polymer forms of actin were characterized. In general, low ionic strength and cold temperatures drive the actin towards the monomer form, while high ionic strength and heat promotes the polymer form.

### Actin ELISA

ELISA formats were evaluated with actin prepared as monomer and polymer forms in order to identify conditions that would allow consistent measurement of one form or another. High ionic strength buffers elicited ELISA signals in some of the antibody paired solid phase and conjugate preparations, while monomer forms of actin (low ionic strength) were not detected in any of the antibody pairs.

To confirm the observation that the polymer but not the monomer form, was detected in the assay, ultra centrifugation studies were conducted.

Equivalent aliquots of purified actin, reconstituted in water at 1 mg/mL, were diluted 10× (20 uL) into 180 uL of either 10 mM Tris, pH 7.2 (low ionic strength) or phosphate buffered saline (PBS, pH 7.2, high ionic strength) in micro centrifuge tubes (Beckman Airfuge) and allowed to equilibrate at ambient temperature for 1 hour. The tubes were centrifuged at 100,000×g for 1 hour. The supernatants containing either monomer actin or no actin (polymerized and in the pellet) were diluted 5× into PBS and diluted for ELISA testing. The residual actin in the centrifuge tubes, of which one theoretically contained a pellet in the high ionic strength preparation and the other having no pellet as the monomer did not sediment, were reconstituted/dissolved in PBS, diluted and tested in the ELISA. The results, as summarized in Table 3 indicate that the high ionic strength buffer promoted actin to 92% polymer which was recovered from the pellet and the supernatant contained actin calculated to be 8% monomer. On the other hand, the supernatant of actin prepared in the low ionic strength buffer, when diluted in a high ionic strength buffer constituted approximately 93% (indicating monomer) and only 7% was found in the reconstituted pellet.

TABLE 3
Ultracentrifuge characterization of buffer types which result
in monomer and polymer forms of actin.
Proportion of Actin in Monomer/Polymer forms by Buffer

| Tris (Low Salt) | | PBS (High Salt) | |
|---|---|---|---|
| Monomer | Polymer | Monomer | Polymer |
| 0.93 +/− 0.06 | 0.07 +/− 0.06 | 0.08 +/− 0.05 | 0.92 +/− 0.05 |

The resulting ELISA format was used for the detection and measurement of human platelet actin as described herein above. Calibration was achieved by using native actin, purified from human platelets (Cytoskeleton). Actin calibrators were prepared using the purified platelet actin. The purified actin was prepared according the to manufacturer's instructions, diluted to 1 mg/ml in water and stored at −80° C. Calibrators were prepared fresh 15 to 60 minutes before plating. Serial dilutions of actin were prepared in “polymerization buffer”: 10 mM Tris, pH 7.5 (Sigma, MP Biomed.), 2 mM MgCl2 (Sigma) and 50 mM KCl (Sigma). To limit variability, a large stock of polymerization buffer was prepared prior to testing, 0.2 um filtered and used throughout testing. Actin calibrator concentrations ranged from 1000 ng/ml to 31 ng/ml. One set of actin calibrators was used for an entire day of testing.

The human platelet samples were lysed in Platelet Lysis buffer as previously described. From the 16× platelet lysate, samples were diluted 2× to achieve a final 32× in standard 2-ml Eppendorf tubes, vortexed briefly (1-2 seconds) and incubated at room temperature for between 1 and 2 hours before plating. The monomer:polymer actin ratio was highly sensitive to vortexing time, incubation time/temperature and buffer composition; since detection by the actin ELISA was dependent on the monomer:polymer ratio, efforts were made to prepare and plate the actin samples in a repeatable manner throughout the entire testing process.

With the use of a fluorescent Actin Polymerization method (Cytoskeleton, Inc) and ultra centrifugation studies, buffer conditions were identified that promote either monomer (low ionic strength, i.e.: 10 mM Tris)) or polymer (high ionic strength, e.g., 0.1 M sodium phosphate, 0.15 M NaCl, pH 7.4), data not shown.

All of the antibodies screened as solid phase and/or biotinylated detectors turned out to detect only the polymer form (data not shown). Without wishing to be bound by theory, actin when used as an immunogen likely polymerizes when injected into a mouse with physiological ionic strength, thus the commercially available antibodies tested tend to recognize only the polymeric form. All subsequent experiments took advantage of the determination that the actin antibodies preferentially bind polymerized actin. Prior to and/or during assay for actin, conditions are established that result in substantially polymerized actin.

Normalization with Model PRP/Platelet Systems and Platelet Pellets Obtained from Normal Subjects.

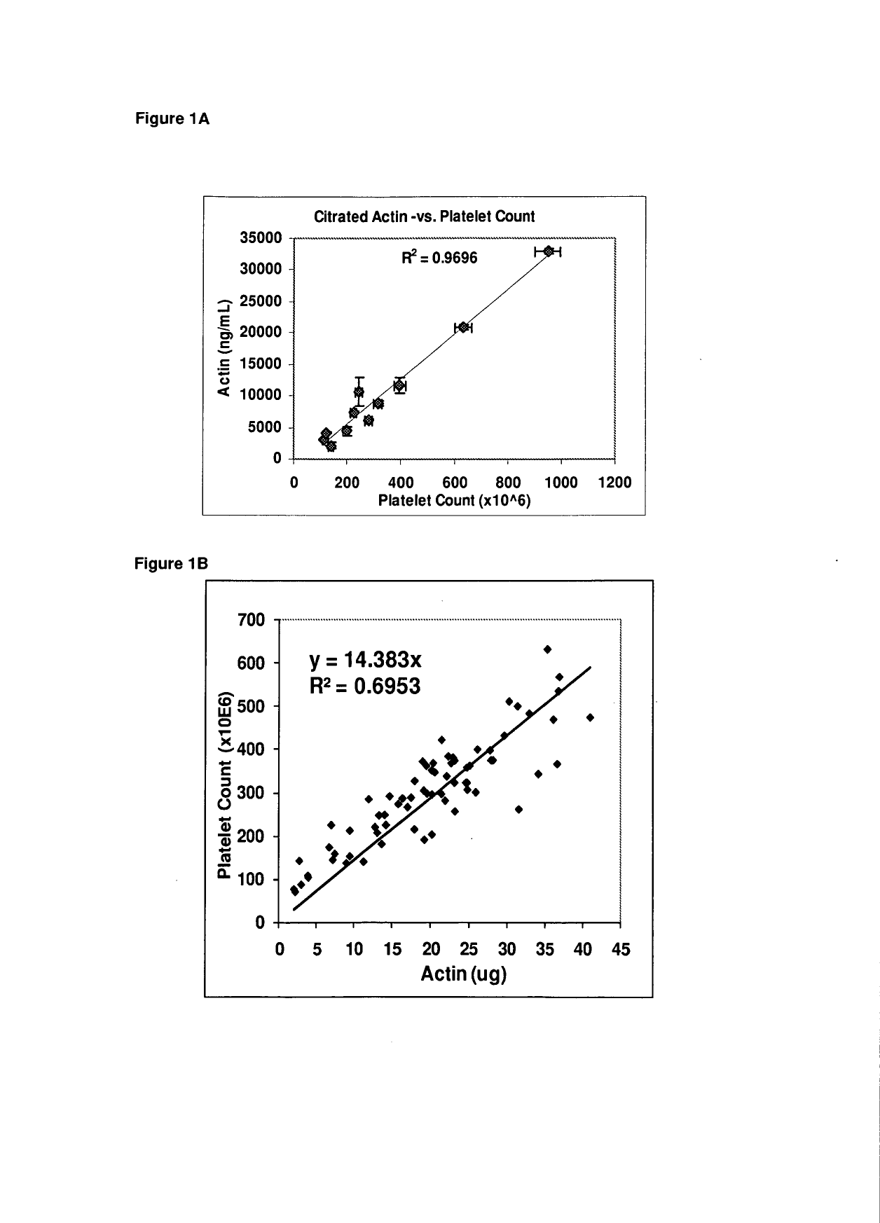

In order to assess the ability of the actin ELISA method to detect and correct for differences in platelet levels, a set of platelet samples was prepared from varying volumes of a Platelet Rich Plasma (PRP) pool, which was subsequently centrifuged to prepare the platelet pellets. The platelet samples were then lysed, diluted and tested in the Actin ELISA assay (FIG. 1). A very good correlation of actin to platelet count (R2=97%) was found.

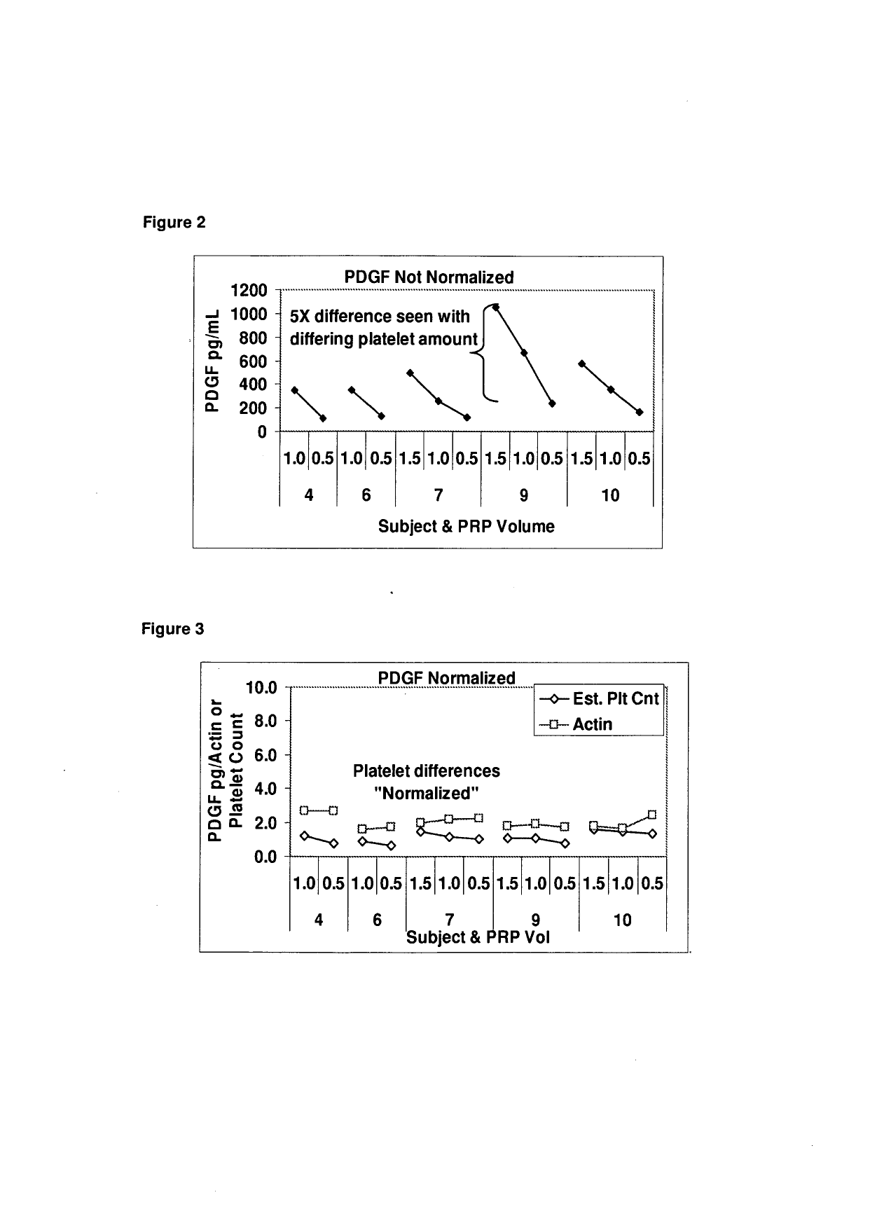

A selected biomarker (PDGF) was tested in the platelets from 5 individuals where the level of platelets was intentionally varied. A wide range of PDGF was detected due to the variance of platelet number (FIG. 2). When the same data was corrected (e.g., normalized, divided by) using the estimated platelet count or the actual actin measurements, (FIG. 3) the difference introduced by varying levels of platelets was no longer a factor.

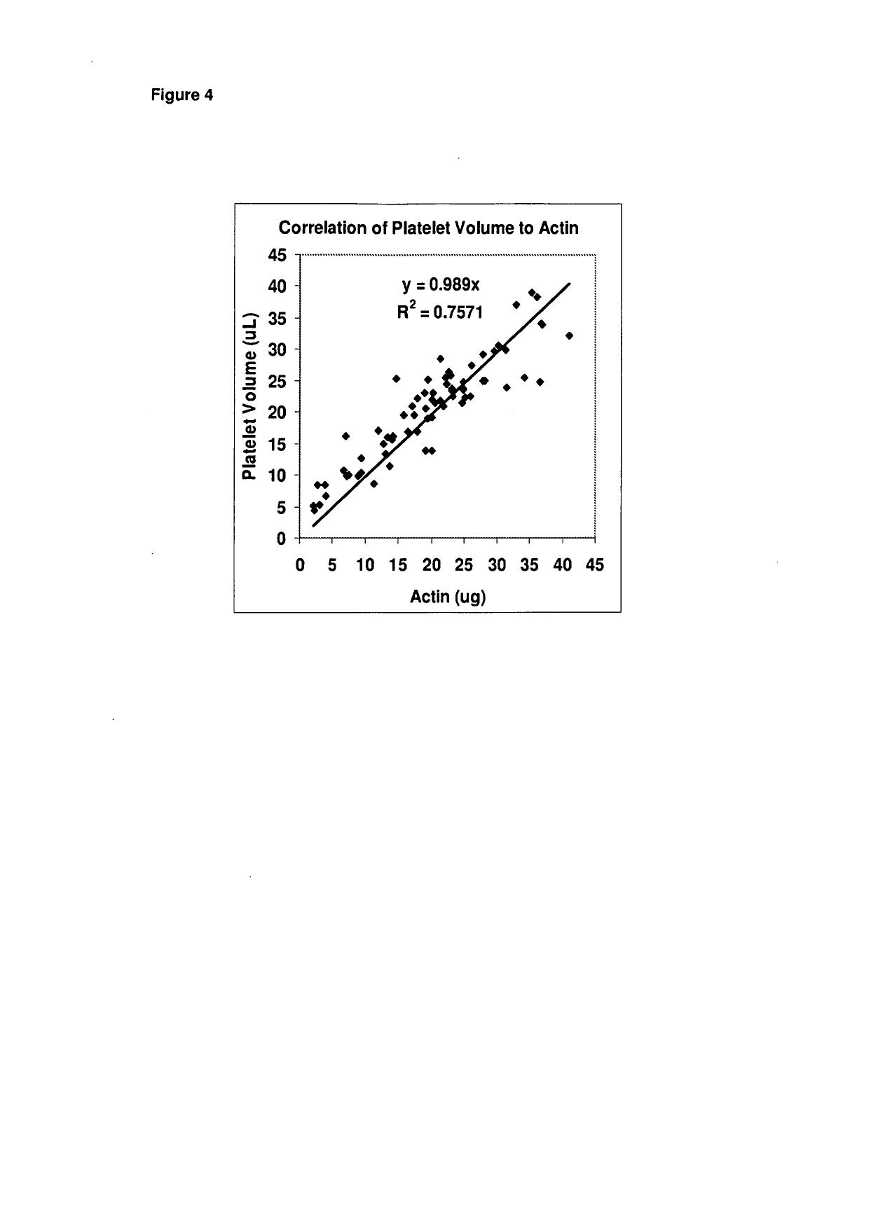

A mathematical relationship useful for clinical assessments between Actin (in ug/mL) and platelet count was devised. CBC platelet counting was performed, including the mean platelet volume, on PRP samples taken from 57 individual samples. The platelet pellet samples were prepared as described earlier and assayed for actin in eight (8) runs, one per set of subject samples, with the Actin ELISA. The Actin results for the 57 samples (Avg 23.11++/−6.53 ug/mL PRP) had a normal distribution (p=0.176). The total corresponding platelet volume measurements (CBC platelet count X CBC mean platelet volume, 23.01+/−7.39 μL/mL PRP) also had a normal distribution (p=0.100). A linear regression relationship was calculated with a resulting equation (Equation 1) and a correlation of 0.757 as depicted in FIG. 4.

The following linear regression based relationship describes the platelet volume relative to Actin.

Y(platelet,uL)=0.989(Actin,ug) Equation 1

Of particular interest for the use of the Actin ELISA as a surrogate marker for platelet counts, is the degree of error introduced into platelet calculations. Table 1 describes the estimate of a platelet count in a given platelet pellet preparation based on a CBC count taken with whole blood of 299,000, with an error range 221,000 to 542,000 with a 60,000 platelet/uL bias and an overall range 321,000 platelets per mL based on the 95% confidence intervals.

In a direct comparison of platelet counts in PRP by CBC and of the platelet volume from platelets prepared from the same PRP, the results are close to one another.

Table 4 shows the results obtained in a comparative study conducted with the PRP obtained from 19 normal subjects. The PRP from each subject was pooled and split into a 1 mL aliquot for CBC counting and a 1 mL aliquot for centrifugation to obtain the platelet pellet. The platelet pellet sample was lysed and tested in the Actin ELISA, as described earlier and converted into platelet count by the relationship described in Equation 1.

TABLE 4
Platelet counts determined by the Actin Method compared to CBC

| | CBC PLT | Actin Method Plt |
|---|---|---|
| ID | Count × 10E3 | Count × 10E3 |
| Avg | 336 | 336 |
| SD | 100 | 80 |
| 95% CI LB | 136 | 176 |
| 95% CI UB | 536 | 495 |
| 95% CI Range | 400 | 319 |

As shown, the averages are the same because this CBC data was used to generate the linear regression equation 1. However, surprisingly the actin normalization method was found to have better precision, as seen by a 95% CI Range of 319, compared to 400 calculated from the results obtained by the CBC method.

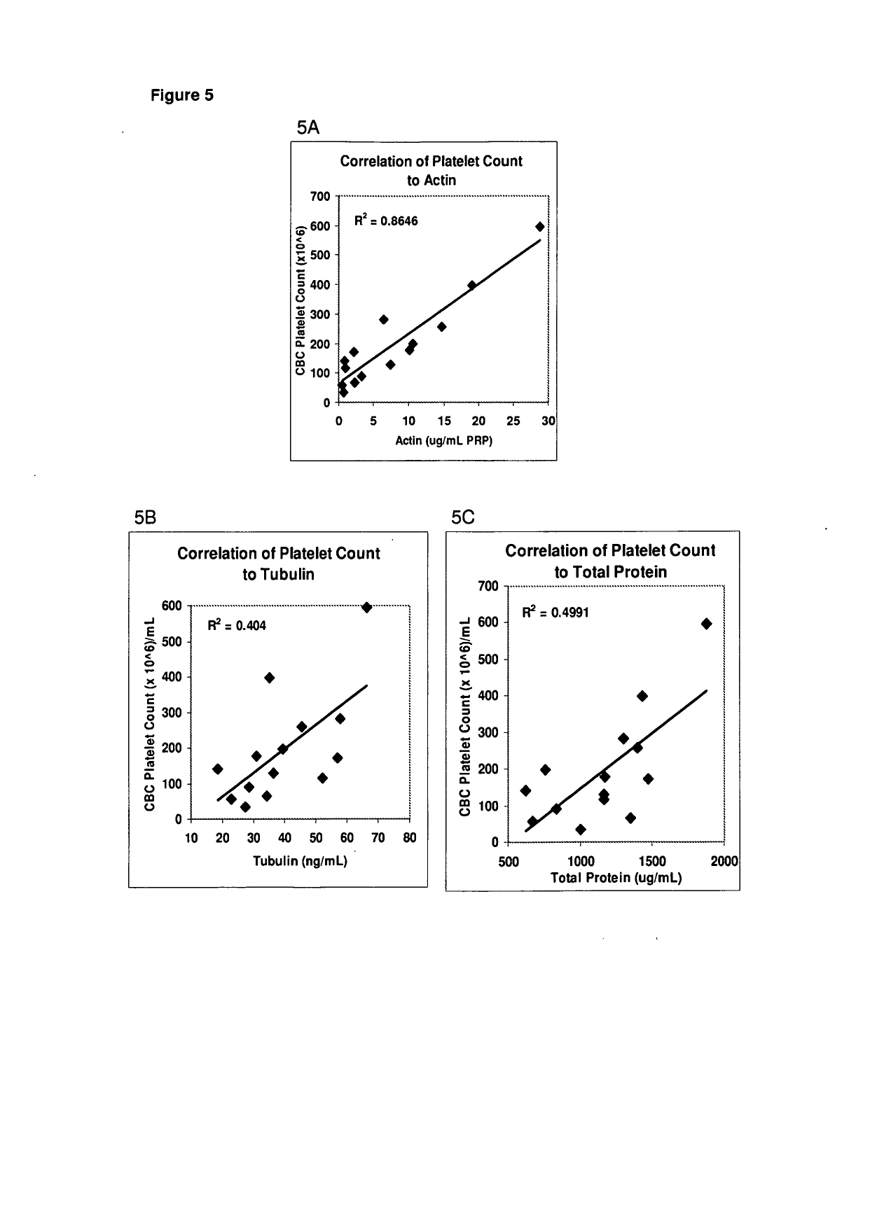

Another relationship was discovered for determining a platelet count in a given sample, relative to a volume, using the actin value and correlating to platelet count, obtained from CBC. This relationship used the same data as that used for Equation 1 and FIG. 4 (Actin correlation to Platelet Volume), but without the volume component (MPV). The correlation (R²) was 0.695 as shown in FIG. 5.

A linear regression relationship was developed in order to describe the platelet count relative to Actin.

Y(Platelet Count/mL×106)=14.383(Actin,ug/mL) Equation 2

### Results: Non-Diseased Subjects (Control)

The total platelet volume found in a given sample was calculated with the linear regression relationship described in Equation 1.

TABLE 5
Normal Ranges. Platelet concentrations relative to the platelet count and
platelet volume (per μL). Plasma concentrations are shown per mL and per μL for
comparison to platelet concentration. X-fold is the difference in concentration as a
ratio between Platelet and Plasma concentrations (in the same units). Min and Max
are defined by the 95% empirical confidence interval (2.5^{th}-97.5^{th} percentile).

| | | | | | | 95% CI Range* | |
|---|---|---|---|---|---|---|---|
| | Matrix | Unit | Avg | Median | SD | Min | Max |
| VEGF | Platelet | pg/10\hat{ }6 | 0.74 | 0.68 | 0.37 | 0.02 | 1.47 |
| | | pg/μL | 11 | 10 | 5.4 | 0.0 | 22 |
| | | nM | 0.24 | 0.22 | 0.12 | 0.00 | 0.48 |
| | Plasma | pg/mL | 46 | 45 | 18 | 11 | 81 |
| | | pg/μL | 0.05 | 0.05 | 0.02 | 0.01 | 0.08 |
| | | pM | 1.0 | 1.0 | 0.4 | 0.2 | 1.8 |
| | X-Fold | pg/μL | | 215 | | | |
| PF-4 | Platelet | ng/10\hat{ }6 | 12 | 10 | 5.0 | 2.4 | 22 |
| | | ng/μL | 178 | 150 | 74 | 34 | 323 |
| | | μM | 5.7 | 4.8 | 2.4 | 1.1 | 10.3 |
| | Plasma | ng/mL | 363 | 291 | 255 | 0 | 862 |
| | | ng/μL | 0.36 | 0.29 | 0.25 | 0.00 | 0.86 |
| | | nM | 11.6 | 9.3 | 8.2 | 0.0 | 27.6 |
| | X-Fold | ng/μL | | 516 | | | |
| PDGF | Platelet | pg/10\hat{ }6 | 23 | 21 | 6 | 12 | 33 |
| | | pg/μL | 330 | 312 | 83 | 167 | 494 |
| | | nM | 12 | 11 | 3.0 | 6.0 | 18 |
| | Plasma | pg/mL | 376 | 341 | 236 | 0 | 838 |
| | | pg/μL | 0.38 | 0.34 | 0.24 | 0.00 | 0.84 |
| | | pM | 13.4 | 12.2 | 8.4 | 0.0 | 29.9 |
| | X-Fold | pg/μL | | 914 | | | |
| TSP-1 | Platelet | ng/10\hat{ }6 | 31 | 27 | 12 | 7 | 54 |
| | | ng/μL | 449 | 403 | 178 | 101 | 798 |
| | | uM | 1.0 | 0.90 | 0.39 | 0.22 | 1.8 |
| | Plasma | ng/mL | 559 | 496 | 272 | 26 | 1092 |
| | | ng/μL | 0.56 | 0.50 | 0.27 | 0.03 | 1.09 |
| | X-Fold | ng/μL | | 813 | | | |
| bFGF | Platelet | pg/10\hat{ }6 | 0.44 | 0.42 | 0.15 | 0.15 | 0.74 |
| | | pg/μL | 6.40 | 6.17 | 2.00 | 2.47 | 10.3 |
| | | nM | 0.34 | 0.33 | 0.11 | 0.13 | 0.55 |
| | Plasma | pg/mL | 365 | 371 | 143 | 86 | 645 |
| | | pg/μL | 0.365 | 0.371 | 0.143 | 0.086 | 0.645 |
| | | pM | 19.5 | 19.8 | 7.6 | 4.6 | 34.5 |
| | X-Fold | pg/μL | | 17 | | | |
| ES | Platelet | pg/10\hat{ }6 | 5.6 | 5.1 | 3.0 | 0.0 | 11.5 |
| | | pg/μL | 81 | 74 | 42 | 0.0 | 163 |
| | | nM | 4.0 | 3.7 | 2.1 | 0.0 | 8.2 |
| | Plasma | pg/mL | 119418 | 110900 | 34345 | 52101 | 186734 |
| | | pg/μL | 119 | 111 | 34 | 52 | 187 |
| | | nM | 6.0 | 5.5 | 1.7 | 2.6 | 9.3 |
| | X-Fold | pg/μL | | 0.7 | | | |

All biomarkers are normalized to actin and expressed per μL platelet volume or 10⁶ platelets.
*X-Fold = Platelet concentration/Plasma Concentration, in same units
*Defined by the 95% empirical confidence interval (2.5th-97.5th percentile)

### Example 3

### Data Used to Derive the Relationship of Platelet Metrics to Actin Measurements

Individual platelet counts, mean platelet volume and actin values used to derive a linear regression relationship of actin to platelet volume and the resultant platelet counts and volumes are provided in the following Table 6.

TABLE 6

| | CBC PLT | | Total PLT | Actin |
|---|---|---|---|---|
| | Count × 10E6 | CBC MPV fL | Vol (uL) | ug/mL |
| ID | per mL PRP | (10E-15 Liter) | per mL PRP | PRP |
| 203 | 481 | 7.7 | 37 | 33 |
| 204 | 280 | 7.5 | 21 | 22 |
| 205 | 467 | 8.2 | 38 | 36 |
| 206 | 430 | 6.9 | 30 | 30 |
| 207 | 190 | 7.3 | 14 | 19 |
| 208 | 630 | 6.2 | 39 | 35 |
| 209 | 509 | 6.0 | 31 | 30 |
| 210 | 373 | 7.8 | 29 | 28 |
| 211 | 294 | 6.5 | 19 | 20 |
| 212 | 246 | 6.5 | 16 | 13 |
| 213 | 498 | 6.0 | 30 | 31 |
| 214 | 321 | 7.4 | 24 | 23 |
| 215 | 202 | 6.9 | 14 | 20 |
| 216 | 382 | 6.4 | 24 | 22 |
| 217 | 180 | 6.3 | 11 | 14 |
| 219 | 211 | 6.0 | 13 | 9 |
| 220 | 373 | 6.7 | 25 | 28 |
| 221 | 296 | 6.4 | 19 | 19 |
| 222 | 379 | 6.8 | 26 | 23 |
| L11 | 366 | 6.3 | 23 | 20 |
| L12 | 303 | 6.8 | 21 | 19 |
| L13 | 356 | 6.6 | 23 | 25 |
| L14 | 372 | 6.3 | 23 | 23 |
| L15 | 321 | 6.7 | 22 | 25 |
| L21 | 420 | 6.8 | 29 | 21 |
| L22 | 360 | 7.0 | 25 | 19 |
| L23 | 325 | 6.8 | 22 | 18 |
| L24 | 287 | 6.8 | 20 | 17 |
| L25 | 398 | 6.9 | 27 | 26 |
| L31 | 285 | 5.9 | 17 | 16 |
| L32 | 370 | 6.2 | 23 | 19 |
| L33 | 350 | 6.3 | 22 | 20 |
| L34 | 360 | 6.2 | 22 | 25 |
| L35 | 345 | 6.2 | 21 | 20 |
| L41 | 206 | 6.5 | 13 | 13 |
| L42 | 139 | 6.2 | 9 | 11 |
| L43 | 247 | 6.3 | 16 | 14 |
| L44 | 219 | 6.8 | 15 | 13 |
| L45 | 244 | 7.2 | 16 | 14 |
| L61 | 321 | 7.7 | 25 | 25 |
| L62 | 336 | 7.6 | 26 | 22 |
| L64 | 272 | 7.2 | 20 | 16 |
| L65 | 366 | 7.2 | 26 | 23 |
| L81 | 533 | 6.4 | 34 | 37 |
| L82 | 364 | 6.8 | 25 | 37 |
| L83 | 396 | 6.3 | 25 | 28 |
| L84 | 566 | 6.0 | 34 | 37 |
| L85 | 472 | 6.8 | 32 | 41 |
| L91 | 299 | 7.5 | 22 | 26 |
| L92 | 305 | 7.8 | 24 | 25 |
| L93 | 341 | 7.5 | 26 | 34 |
| L94 | 295 | 7.4 | 22 | 21 |
| L95 | 265 | 7.9 | 21 | 17 |
| LA1 | 260 | 9.2 | 24 | 32 |
| LA2 | 255 | 8.8 | 22 | 23 |
| LA3 | 290 | 8.7 | 25 | 15 |
| LA4 | 214 | 7.9 | 17 | 18 |
| 422 | 70 | 6.2 | 4 | 2 |
| 522 | 158 | 6.4 | 10 | 7 |
| 842 | 283 | 6.0 | 17 | 12 |
| 843 | 142 | 6.0 | 8 | 3 |
| 242 | 144 | 6.8 | 10 | 7 |
| 451 | 224 | 7.2 | 16 | 7 |
| A42 | 107 | 7.9 | 8 | 4 |
| 122 | 152 | 6.8 | 10 | 9 |
| 123 | 76 | 6.8 | 5 | 2 |
| 642 | 136 | 7.2 | 10 | 9 |
| 352 | 173 | 6.2 | 11 | 7 |
| 353 | 86 | 6.2 | 5 | 3 |
| 412 | 103 | 6.5 | 7 | 4 |
| Avg | 300 | 6.9 | 20.6 | 19.8 |
| SD | 120 | 0.7 | 8.1 | 9.5 |

1. An assay for determining the level of a biomarker in a platelet preparation comprising:
(a) determining the level of a surrogate marker in a platelet preparation sample, wherein the surrogate marker corresponds to platelet number, platelet concentration or platelet volume;
(b) determining the level of a biomarker in the sample,
(c) normalizing the level of the biomarker in the sample to the level of the surrogate marker, whereby a normalized biomarker level for the sample is determined.

2. The assay of claim 1, wherein the normalizing step comprises dividing the value obtained for the level of the biomarker in the sample by the value obtained for the level of the surrogate marker.

3. The assay of claim 1, wherein the surrogate marker is polymerized or monomeric actin.

4. The assay of claim 1, wherein step (a) comprises placing the sample obtained from an individual under conditions that induce actin polymerization, such that actin in the sample is substantially polymerized.

5. (canceled)

6. The assay of claim 1, wherein the biomarker is an angiogenic regulator.

7. A method for identifying a surrogate marker for platelet number, platelet concentration, or platelet volume, the method comprising:
(a) assaying the amount of a plurality of candidate markers in each sample of a series of samples prepared from a single platelet preparation according to a sampling factor;
(b) comparing the amount of each candidate marker in each sample to the amount of candidate marker predicted according to the sampling factor, wherein the comparing step identifies the candidate marker of the plurality assayed in step (a) that has the closest correlation between the amount of candidate marker predicted and the amount of candidate marker measured, whereby the candidate marker is identified as a surrogate marker for platelet number, platelet concentration, or platelet volume.

8. The method of claim 7, wherein said platelet preparation comprises lysed platelets.

9. The method of claim 7, further comprising testing the identified surrogate marker for variation under different physiological conditions.

10. A method for normalizing the amount of a biomarker in a sample, the method comprising normalizing the amount of a biomarker measured in a platelet preparation relative to a surrogate marker identified using the method of claim 7.

11. (canceled)

12. (canceled)

13. (canceled)

14. (canceled)

15. A method for assessing a change in biomarker level of an individual, the method comprising:
(a) placing a sample of isolated platelets obtained from said individual under conditions that induce actin polymerization, such that actin in said sample is substantially polymerized;
(b) contacting said sample with an agent that selectively binds polymerized actin and detecting formation of a complex between said agent and polymerized actin, whereby the level of actin in said sample is measured;
(c) measuring the level of a biomarker in said sample;
(d) normalizing the level of said biomarker in said sample to the measured level of polymerized actin in said sample,
(e) comparing a normalized level of said biomarker in said sample to a reference, and if a difference in the normalized level of said biomarker compared to said reference is identified, a change in the level of said biomarker of said individual is identified.

16. The method of claim 15, wherein said conditions that induce actin polymerization comprise a high concentration of salt.

17. The method of claim 15, wherein the biomarker is an angiogenic regulator, and wherein a change in the level of the angiogenic regulator is indicative of a change in angiogenic state and/or the presence of an angiogenic disorder.

18. (canceled)

19. The method of claim 17, wherein said angiogenic disorder is a tumor associated disease.

20. The method of claim 15, wherein said reference is obtained from biological samples obtained from a population of individuals.

21. (canceled)

22. (canceled)

23. A kit for detecting a normalized level of at least one biomarker in platelets, the kit comprising:
(a) at least one reagent which, when contacted with an isolated platelet sample induces actin polymerization or depolymerization; and
(b) an agent that selectively binds either polymerized actin where the reagent of step (a) induces actin polymerization, or monomeric actin where the reagent of step (a) induces actin depolymerization;
(c) an agent that binds a biomarker; and
(d) packing materials and instructions for normalizing the level of the at least one biomarker to the level of polymerized or monomeric actin.

24. The kit of claim 23, further comprising a solid support.

25. The kit of claim 23, further comprising a reagent that generates a detectable signal.

26. (canceled)

27. The kit of claim 23, further comprising an agent that binds at least one other biomarker.

28. A computer readable storage medium having computer readable instructions recorded thereon to define software modules for implementing on a computer a method for assessing a biomarker level in a platelet sample, said computer readable storage medium comprising:
(a) instructions for storing and accessing data representing a level of a biomarker and a level of a surrogate marker determined for a sample of isolated platelets obtained from at least one individual;
(b) instructions for normalizing said level of said biomarker to said level of said surrogate marker via a normalization module, thereby producing a normalized level of said biomarker,
(c) instructions for displaying retrieved content to a user, wherein the retrieved content comprises a normalized biomarker level.

29. The computer readable storage medium of claim 28, further comprising instructions for comparing said normalized level of said biomarker to reference data stored on said storage device using a comparison module, whereby a change in the biomarker level is determined.

30. (canceled)

31. (canceled)

32. (canceled)

33. (canceled)
